(19) United States
(12) Patent Application Publication
(10) Pub. No.: US 2025/0222178 A1
(43) Pub. Date: Jul. 10, 2025

(54) ANTIBACTERIAL BIOMEDICAL IMPLANTS AND ASSOCIATED MATERIALS, APPARATUS, AND METHODS

(71) Applicant: Sintx Technologies, Inc., Salt Lake City, UT (US)

(72) Inventors: Bryan J. McEntire, Salt Lake City, UT (US); Bhajanjit Singh Bal, Salt Lake City, UT (US); Ryan M. Bock, Salt Lake City, UT (US)

(21) Appl. No.: 19/017,633

(22) Filed: Jan. 11, 2025

Related U.S. Application Data
(63) Continuation of application No. 17/384,236, filed on Jul. 23, 2021, now abandoned.
Application No. 17/384,236 is a continuation-in-part of application No. 17/029,534, filed on Sep. 23, 2020, now Pat. No. 11,738,122.
Application No. 17/029,534 is a continuation of application No. 15/470,637, filed on Mar. 27, 2017, now Pat. No. 10,806,831.
Application No. 15/470,637 is a continuation-in-part of application No. 13/890,876, filed on May 9, 2013, now abandoned.
(60) Provisional application No. 61/644,906, filed on May 9, 2012.

(51) Int. Cl. A61L 31/02; A61L 27/06; A61L 27/30; A61L 27/50; A61L 31/08; A61L 31/12; A61L 31/14; A61L 31/16

(52) U.S. Cl. CPC A61L 31/028; A61L 27/06; A61L 27/306; A61L 27/50; A61L 31/022; A61L 31/026; A61L 31/088; A61L 31/12; A61L 31/14; A61L 31/16; A61L 2300/10; A61L 2300/406; A61L 2400/18; A61L 2430/02; A61L 2430/38

(57) ABSTRACT

Methods for improving the antibacterial and/or bone-forming characteristics of biomedical implants and related implants manufactured according to such methods. In some implementations, a biomedical implant may comprise a composite of a silicon nitride ceramic powder dispersed within a poly-ether-ether-ketone (PEEK) or a poly-ether-ketone-ketone (PEKK) substrate material. In some implementations, the biomedical implant may be 3D printed.

### CROSS REFERENCE TO RELATED APPLICATIONS

This application is a continuation of U.S. patent application Ser. No. 17/384,236 filed Jul. 23, 2021, which is a Continuation-In-Part of U.S. patent application Ser. No. 17/029,534 filed Sep. 23, 2020, now U.S. Pat. No. 11,738,122 which is a continuation of Ser. No. 15/470,637 filed Mar. 27, 2017, now U.S. Pat. No. 10,806,831, which is a Continuation-In-Part of U.S. Non-Provisional application Ser. No. 13/890,876, filed on May 9, 2013, which claims benefit of U.S. Provisional Patent Application No. 61/644,906 filed May 9, 2012, which are herein incorporated herein by reference in their entirety.

### FIELD

The present disclosure generally relates to antibacterial biomedical implants, and in particular to materials, apparatuses and methods for improving the antibacterial characteristics of an intervertebral spinal implant.

### BACKGROUND

Polymeric materials are poor at both osteointegration and microbial resistance. Prior work to overcome this involves inclusion of either an antimicrobial or osteogenic materials onto or into the polymeric material. In these cases, hydroxyapatite is typically cited as the material that improves osteoconduction, whereas the antimicrobial compound is typically silver or an antibiotic. However, there is a need for the improvement of the osteogenic and anti-infective properties of the polymeric material using one material.

Craniomaxillofacial (CMF) implants are used for head and facial reconstruction due to trauma, infection, cancer, and congenital and developmental deformities. To be effective, prosthetic devices must be biocompatible, infection resistant, strong, durable, thermally insulating, shape stable, osteoconductive, low cost, and readily available. Current CMF implants lack bioactivity, infection control, osseous integration, mechanical stability, or radiographic imaging. There is thus a need for medical imaging compatible 3D printed biomaterials for CMF osteoplasty that can be personalized, promote integration, and prevent infection.

It is with these observations in mind, among others, that various aspects of the present disclosure were conceived and developed.

### SUMMARY

A need exists for an improved biomedical implant with antibacterial properties. Accordingly, one embodiment of the present disclosure may include a method for improving the antibacterial characteristics of a biomedical implant. The method may include the steps of providing a biomedical implant and loading the biomedical implant with about 10% to about 20% of a powder, wherein the powder comprises a silicon nitride material. The method may further include increasing the surface roughness of at least a portion of the biomedical implant to a roughness profile having an arithmetic average of at least about 500 nm Ra to improve the antibacterial characteristics of the biomedical implant by at least one of micromachining, grinding, polishing, laser etching, laser texturing, sand- or other abrasive-blasting, chemical etching, thermal etching, and plasma etching. The silicon nitride material may be selected from the group consisting of α-Si₃N₄, β-Si₃N₄, β-SiYAlON, and combinations thereof. The biomedical implant may be a hip implant, intervertebral spinal implant, bone screw, or a craniomaxillofacial implant. In one example, the biomedical implant may be a silicon nitride coating on a titanium femoral stem of a hip implant. Examples of intervertebral spinal implants may include cervical or lumbar devices. Similarly, examples of craniomaxillofacial implants may include burr plugs, cranial, temporal, maxilla, or zycomatic, and mandible plates and screws, and temporal mandibular joints. The biomedical implant may be a bone screw. The biomedical implant may include poly-ether-ether-ketone (PEEK) or titanium, and α- or β-Si₃N₄ powder, titanium or PEEK and β-SiYAlON powder, poly-ether-ketone-ketone (PEKK), and α- or β-Si₃N₄ powder, or PEKK and β-SiYAlON powder.

In other embodiments, the method may further include applying a coating of silicon nitride to the biomedical implant. The step of increasing a surface roughness of at least a portion of the biomedical implant may be performed either prior to or after the step of applying a coating to the biomedical implant, and the step of increasing a surface roughness of at least a portion of the biomedical implant may include increasing a surface roughness of at least a portion of the coating. The step of increasing a surface roughness of at least a portion of the biomedical implant to a roughness profile having an arithmetic average of at least about 1,250 nm Ra or between about 2,000 nm Ra and about 5,000 nm Ra.

Another implementation of the present disclosure may take the form of a biomedical implant with improved antibacterial characteristics. The biomedical implant may include a polymeric or metallic substrate material; and about 10% volume percent (vol. %) to about 20 vol. % of a powder, wherein the powder comprises a silicon nitride material. At least a portion of the implant may have an increased surface roughness profile having an arithmetic average of at least about 500 nm Ra created by at least one of micromachining, grinding, polishing, laser etching, laser texturing, sand- or other abrasive-blasting, chemical etching, thermal etching, and plasma etching. The silicon nitride material may be selected from the group consisting of α-Si₃N₄, β-Si₃N₄, β-SiYAlON, and combinations thereof. The substrate material may include poly-ether-ether-ketone (PEEK), poly-ether-ketone-ketone (PEKK), or titanium. The biomedical implant may be an intervertebral spinal implant, a hip implant, a bone screw, or a craniomaxillofacial implant. The biomedical implant may include a hip implant with a silicon nitride coating on a femoral stem of the hip implant. The biomedical implant may further include a silicon nitride coating on the biomedical implant.

Further provided herein is a method for forming a biomedical implant. In some embodiments, the method includes the steps of: dispersing silicon nitride powder within a poly-ether-ether-ketone (PEEK) or poly-ether-ketone-ketone (PEKK) substrate material to form a composite material; and forming the composite material into a biomedical implant. The biomedical implant has improved antibacterial characteristics and/or improved bone-forming characteristics as compared to a monolithic PEEK or PEKK implant.

Also provided herein is a biomedical implant that includes a poly-ether-ether-ketone (PEEK) or a poly-ether-ketone-ketone (PEKK) substrate material; and a powder comprising α-Si₃N₄, β-Si₃N₄, β-SiYAlON, or combinations thereof. In various embodiments, the powder is dispersed within the substrate material, forming a composite material.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The written disclosure herein describes illustrative embodiments that are non-limiting and non-exhaustive. Reference is made to certain of such illustrative embodiments that are depicted in the figures, in which:

FIG. 1A is a perspective view of one embodiment of a spinal implant; FIG. 1B is a perspective view of the spinal implant of FIG. 1A after a surface roughening process has been applied to the implant; and FIG. 1C is a perspective view of the spinal implant of FIG. 1B with surface features for minimizing implant migration, according to one aspect of the present disclosure.

FIG. 2A is a perspective view of another embodiment of a spinal implant having a coating applied thereto; and FIG. 2B is a perspective view of the embodiment of FIG. 2A after a surface roughening process has been applied to the coating of the implant, according to one aspect of the present disclosure.

FIG. 3A is a perspective view of an embodiment of a hip stem implant having a coating applied to a portion of the implant; and FIG. 3B is a perspective view of the embodiment of FIG. 3A after a surface roughening process has been applied to the coating of the implant, according to one aspect of the present disclosure.

FIG. 4A is a cross-sectional view taken along line 4A-4A in FIG. 3A; and FIG. 4B is a cross-sectional view taken along line 4B-4B in FIG. 3B.

FIG. 5A is a perspective view of an embodiment of a bone screw implant; and FIG. 5B is a perspective view of the embodiment of FIG. 5A after a surface roughening process has been applied to the implant, according to one aspect of the present disclosure.

FIG. 6A shows fluorescence spectroscopy images of SaOS-2 cells on monolithic PEEK; FIG. 6B shows fluorescence spectroscopy images of SaOS-2 cells on PEEK with 15% vol. % α-Si₃N₄; FIG. 6C shows fluorescence spectroscopy images of SaOS-2 cells on PEEK with 15% vol. % β-Si₃N₄; and FIG. 6D shows fluorescence spectroscopy images of SaOS-2 cells on PEEK with 15 vol. % β-SiYAlON, according to one aspect of the present disclosure.

FIG. 7 is a graph of the results of cell counting based on the fluorescence microscopy, according to one aspect of the present disclosure.

FIG. 8A shows SEM images of monolithic PEEK before and after exposure to the SaOS-2 cells; FIG. 8B shows SEM images of PEEK with 15% vol. % α-Si₃N₄ before and after exposure to the SaOS-2 cells; FIG. 8C shows SEM images of PEEK with 15% vol. % β-Si₃N₄ before and after exposure to the SaOS-2 cells; and FIG. 8D shows SEM images of PEEK with 15% vol. % β-SiYAlON before and after exposure to the SaOS-2 cells, according to one aspect of the present disclosure.

FIG. 9 is a graph of the results of 3D laser microscopy of the substrate materials, showing the bony apatite volume, according to one aspect of the present disclosure.

FIG. 10A is a Raman microprobe spectroscopy image of β-SiYAlON filled PEEK after 7 days of being exposed to SaOS-2 cells; and FIG. 10B is graph of Raman intensity of β-SiYAlON filled PEEK after 7 days of being exposed to SaOS-2 cell, according to one aspect of the present disclosure.

FIG. 11A shows fluorescence microscopy images with DAPI/CFDA staining of S. epidermis on monolithic PEEK; FIG. 11B shows fluorescence microscopy images with DAPI/CFDA staining of S. epidermis on PEEK with 15% vol. % α-Si₃N₄; FIG. 11C shows fluorescence microscopy images with DAPI/CFDA staining of S. epidermis on PEEK with 15% vol. % β-Si₃N₄; and FIG. 11D shows fluorescence microscopy images with DAPI/CFDA staining of S. epidermis on PEEK with 15% β-SiYAlON, according to one aspect of the present disclosure.

FIG. 12 is a graph of the results of CFDA/DAPI stained positive cells on the various substrates, according to one aspect of the present disclosure.

FIG. 13 is a graph of the results of the WST assay (absorbance at 450 nm) for each of the substrates, according to one aspect of the present disclosure.

FIG. 14A shows a design of the lattice structure of a 3D printed cervical cage, according to one aspect of the present disclosure; FIG. 14B shows a design of the teeth of a 3D printed cervical spinal cage, according to one aspect of the present disclosure; FIG. 14C shows a design of the inner/outer-body shell and support hole of a 3D printed cervical spinal cage, according to one aspect of the present disclosure; FIG. 14D shows design of a cross-section of a 3D printed cervical spinal cage, according to one aspect of the present disclosure; FIG. 14E shows a design of a complete 3D printed cervical spinal cage, according to one aspect of the present disclosure.

FIG. 15 is a graph of the water-soluble tetrazolium (WST) absorbance as measured after 0 and 24 hours of in vitro testing with KUSA-A1 cells on titanium, silicon nitride, PEEK, and silicon nitride-PEEK substrates, according to one aspect of the present disclosure.

FIG. 16A is an ALP enzyme stained micrograph of a silicon nitride substrate obtained after 10 days of exposure in osteogenic medium, according to one aspect of the present disclosure; FIG. 16B is an ALP enzyme stained micrograph of a silicon nitride-reinforced PEEK substrate obtained after 10 days of exposure in osteogenic medium, according to one aspect of the present disclosure; FIG. 16C is an ALP enzyme stained micrograph of a titanium alloy substrate obtained after 10 days of exposure in osteogenic medium, according to one aspect of the present disclosure; FIG. 16D is an ALP enzyme stained micrograph of a PEEK substrate obtained after 10 days of exposure in osteogenic medium, according to one aspect of the present disclosure.

FIG. 17 is a graph of the quantitative assessment of ALP enzyme concentrations.

FIG. 18A is an optical image of a titanium alloy substrate after 240 h of in vitro testing with KUSA-A1 cells. FIG. 18B is an optical image of a silicon nitride substrate after 240 h of in vitro testing with KUSA-A1 cells. FIG. 18C is an optical image of a PEEK substrate after 240 h of in vitro testing with KUSA-A1 cells. FIG. 18D is an optical image of a silicon nitride-reinforced PEEK substrate after 240 h of in vitro testing with KUSA-A1 cells. The presence of bone tissue in FIGS. 18A-18D has been identified with red coloring added by software.

FIG. 19 is a graph of the specific volume of hydroxyapatite (μm³/μm²) for titanium alloy, silicon nitride, PEEK, and silicon nitride-reinforced PEEK, as measured by laser microscopy.

FIG. 20A is a false-color EDS-SEM image obtained of a titanium substrate after 10 days of treatment. FIG. 20B is a false-color EDS-SEM image obtained of a silicon nitride substrate after 10 days of treatment. FIG. 20C is a false-color EDS-SEM image obtained of a PEEK substrate after 10 days of treatment. FIG. 20D is a false-color EDS-SEM image obtained of a silicon nitride-reinforced PEEK substrate after 10 days of treatment. In each of FIGS. 20A-20D, green marks the presence of exposed titanium or silicon, red marks the presence of calcium, and blue marks the presence of carbon.

FIG. 21 shows a graph of the FTIR spectra associated with a) silicon nitride; b) PEEK; c) silicon nitride-reinforced PEEK; and d) titanium alloy substrates after testing with KUSA-A1.

FIG. 22 shows a graph of the representative average FTIR spectra from 400 to 2000 cm⁻¹ associated with PEEK (black) and silicon nitride-reinforced PEEK (red) after biological testing with KUSA-A1.

FIG. 23 shows a graph of the representative FTIR average spectra from 400 to 2000 cm⁻¹ associated with silicon nitride (black) and titanium alloy (red) after biological testing with KUSA-A1

FIG. 24A, FIG. 24B, and FIG. 24C show a procedure for preparing a porous PEEK surface embedded with silicon nitride.

FIG. 25A, FIG. 25B, and FIG. 25C show optical and laser microscopy images of the surfaces of a smooth PEEK control sample with a smooth surface (FIG. 25A), a PEEK sample functionalized with coarse NaCl grains (FIG. 25B); and a PEEK sample incorporating the NaCl-silicon nitride mixture (FIG. 25C).

FIG. 26A shows a graph of the surface roughness on a PEEK substrate, a NaCl-PEEK substrate, and a NaCl-silicon-nitride-PEEK substrate. FIG. 26B shows the average FTIR spectra recorded in the spectral interval 400-1100 cm⁻¹ collected on the surface of the NaCl-PEEK sample (upper spectrum) and NaCl-silicon-nitride-PEEK sample (lower spectrum).

FIG. 27A shows a fluorescence spectroscopy image of blue-stained SaOS-2 cell nuclei on a PEEK substrate. FIG. 27B shows a fluorescence spectroscopy image of blue-stained SaOS-2 cell nuclei on a NaCl-PEEK substrate. FIG. 27C shows a fluorescence spectroscopy image of blue-stained SaOS-2 cell nuclei on a NaCl-silicon-nitride-PEEK substrate.

FIG. 28 shows a graph of the area covered by the osteoblasts in FIGS. 27A-27C as a percent and the lactate dehydrogenase (LDH) fractions in cytotoxicity tests.

FIG. 29A shows a SEM image with an inset of an EDX map of a PEEK substrate after one-week exposure to SaOS-2 cells. FIG. 29B shows a SEM image with an inset of an EDX map of a NaCl-PEEK substrate after one-week exposure to SaOS-2 cells. FIG. 29C shows a SEM image with an inset of an EDX map of a NaCl-silicon-nitride-PEEK substrate after one-week exposure to SaOS-2 cells. In the EDX maps, areas with green, blue, and red colors indicate phosphorous, calcium, and carbon, respectively.

FIG. 30 shows a graph of the average FTIR spectra in the region 900-1200 cm⁻¹ detected on the surfaces shown in FIGS. 29A-29C.

FIG. 31 shows a graph of the FTIR absorbance spectrum in the spectral region 1350-1750 cm⁻¹ for a PEEK substrate, a NaCl-PEEK substrate, and a NaCl-silicon-nitride-PEEK substrate.

FIG. 32A shows a graph of the mineral-to-matrix ratio computed from the ratio of the apatite Band 4 (at 1025 cm⁻¹) and the Amide II Band 17 (at 1642 cm⁻¹ from the spectra in FIG. 31. FIG. 32B shows a graph of the calcium to phosphorus ratio from the EDX elemental data for a PEEK substrate, a NaCl-PEEK substrate, and a NaCl-silicon-nitride-PEEK substrate.

FIG. 33A shows a graph of the OD measurement of PEEK, NaCl-PEEK, and NaCl-silicon-nitride-PEEK samples contaminated with S. epidermis bacteria at 12 h, 24 h, and 48 h. FIG. 33B shows a graph of the CFU count on samples contaminated with S. epidermis bacteria at 12 h, 24 h, and 48 h. In both figures, purple (left) represents the PEEK substrate, blue (middle) represents the NaCl-PEEK substrate, and green (right) represents the NaCl-silicon-nitride-PEEK substrate.

FIG. 34A shows a graph of CFU count after autoclaving NaCl-PEEK and NaCl-silicon-nitride-PEEK substrates for 1 hour at 121° C. FIG. 34B shows EDX spectra before autoclaving NaCl-PEEK and NaCl-silicon-nitride-PEEK substrates. FIG. 34C shows EDX spectra after autoclaving NaCl-PEEK and NaCl-silicon-nitride-PEEK substrates.

### DETAILED DESCRIPTION

Embodiments described herein may be best understood by reference to the drawings, wherein like parts are designated by like numerals throughout. It will be readily understood that the components of the present disclosure, as generally described and illustrated in the drawings herein, could be arranged and designed in a wide variety of different configurations. Thus, the following more detailed description of the embodiments of the apparatus is not intended to limit the scope of the disclosure, but is merely representative of possible embodiments of the disclosure. In some cases, well-known structures, materials, or operations are not shown or described in detail.

As used herein, the terms “comprising,” “having,” and “including” are used in their open, non-limiting sense. The terms “a,” “an,” and “the” are understood to encompass the plural as well as the singular. Thus, the term “a mixture thereof” also relates to “mixtures thereof.”

As used herein, “about” refers to numeric values, including whole numbers, fractions, percentages, etc., whether or not explicitly indicated. The term “about” generally refers to a range of numerical values, for instance, 0.5-1%, 1-5% or ±5-10% of the recited value, that one would consider equivalent to the recited value, for example, having the same function or result.

As used herein, the term “silicon nitride” includes Si₃N₄, alpha- (α) or beta-phase (β) Si₃N₄, SiYAlON, SiYON, SiAlON, or combinations of these phases or materials.

Various embodiments of apparatus, methods, and systems are disclosed herein that relate to biomedical implants having antibacterial characteristics and materials and methods for improving the antibacterial function and/or characteristics of such implants. In preferred embodiments, silicon nitride ceramics, composite polymer silicon nitride, or metal silicon nitride implants are provided that may be, in some embodiments, treated so as to improve upon their antibacterial characteristics and/or other desirable characteristics. For example, embodiments and implementations disclosed herein may result in improved inhibition of bacteria adsorption and biofilm formation, improved protein adsorption, and/or enhanced osteoconductive and osteointegration characteristics. Such embodiments may comprise a silicon nitride ceramic or doped silicon nitride ceramic substrate. Alternatively, such embodiments may comprise a silicon nitride or doped silicon nitride coating on a substrate of a different material. In other embodiments, the implant may be a composite, made up of a silicon nitride material and a polymer, or a silicon nitride material and a metal. In still other embodiments, one or more portions or regions of an implant may include a silicon nitride material and/or a silicon nitride coating, and other portions or regions may include other biomedical materials.

The terms used in this specification generally have their ordinary meanings in the art, within the context of the disclosure, and in the specific context where each term is used. Alternative language and synonyms may be used for any one or more of the terms discussed herein, and no special significance should be placed upon whether or not a term is elaborated or discussed herein. In some cases, synonyms for certain terms are provided. A recital of one or more synonyms does not exclude the use of other synonyms. The use of examples anywhere in this specification including examples of any terms discussed herein is illustrative only, and is not intended to further limit the scope and meaning of the disclosure or of any example term.

As another alternative, silicon nitride or other similar ceramic materials may be incorporated into other materials used to form biomedical implants, including but not limited to hip implants, intervertebral spinal cervical or lumbar implants, or craniomaxillofacial implants. For example, silicon nitride may be used as a filler or otherwise incorporated into polymers or biodegradable polymers, such as poly-ether-ether-ketone (PEEK), poly(methyl methacrylate), poly(ethylene terephthalate), poly(dimethylsiloxane), poly(tetrafluoroethylene), polyacrylic acids, polylactic acids, polycarbonates, polyethylene, and/or polyurethane, in their porous scaffolds or bulk structures. In some embodiments, the silicon nitride filler may be β-silicon nitride and may be present in the biomedical implant in amounts ranging from about 1 vol. % to about 99 vol. %. For example, a β-silicon powder may be incorporated into a PEEK biomedical implant in an amount from about 10 vol. % to about 20 vol. %. Silicon nitride may also be used as a filler otherwise incorporated into other materials used to form other biomedical implants, such as metals, including titanium, silver, nitinol, copper, cobalt/chromium, and related alloys, for example. As still another alternative, silicon nitride may be used as a filler or otherwise incorporated into other materials, such as other oxide ceramics and cermets.

In embodiments including one or more coatings, the coating(s) can be applied by any number of methods such as chemical vapor deposition (CVD), physical vapor deposition (PVD), plasma spraying, electro-deposition or electrophoretic deposition, slurry coating and high-temperature diffusion, or any other application method known by those skilled in the art. In some embodiments, the coating thickness can range from between about 5 nanometers up to about 5 millimeters. In other such embodiments, the coating thickness may be between about 1 micrometer and about 125 micrometers. The coating may adhere to the surface of the implant but need not necessarily be hermetic.

Silicon nitride ceramics have tremendous flexural strength and fracture toughness. In some embodiments, such ceramics have been found to have a flexural strength greater than about 700 Mega-Pascal (MPa). Indeed, in some embodiments, the flexural strength of such ceramics have been measured at greater than about 800 MPa, greater than about 900 MPa, or about 1,000 MPa. The fracture toughness of silicon nitride ceramics in some embodiments exceeds about 7 Mega-Pascal root meter (MPa·m^{1/2}). Indeed, the fracture toughness of such materials in some embodiments is about 7-10 MPa·m^{1/2}.

Examples of suitable silicon nitride materials are described in, for example, U.S. Pat. No. 6,881,229, titled “Metal-Ceramic Composite Articulation,” which is incorporated by reference herein. In some embodiments, dopants such as alumina (Al₂O₃), yttria (Y₂O₃), magnesium oxide (MgO), and strontium oxide (SrO), can be processed to form a doped composition of silicon nitride. In embodiments comprising a doped silicon nitride or another similar ceramic material, the dopant amount may be optimized to achieve the highest density, mechanical, and/or antibacterial properties. In further embodiments, the biocompatible ceramic may have a flexural strength greater than about 900 MPa, and a toughness greater than about 9 MPa·m^{1/2}. Flexural strength can be measured on standard 3-point bend specimens per American Society for Testing of Metals (ASTM) protocol method C-1161, and fracture toughness can be measured using single edge notched beam specimens per ASTM protocol method E399. In some embodiments, powders of silicon nitride may be used to form the ceramic implants, either alone or in combination with one or more of the dopants referenced above.

Other examples of suitable silicon nitride materials are described in U.S. Pat. No. 7,666,229 titled “Ceramic-Ceramic Articulation Surface Implants,” which is hereby incorporated by reference. Still other examples of suitable silicon nitride materials are described in U.S. Pat. No. 7,695,521 titled “Hip Prosthesis with Monoblock Ceramic Acetabular Cup,” which is also hereby incorporated by reference.

Silicon nitride has been discovered to have unexpected antibacterial properties and increased bone formation properties. Indeed, as discussed in greater detail below, it has been recently demonstrated that the adhesion and growth of bacteria on silicon nitride materials is substantially reduced with respect to other common spinal implant materials, such as titanium and polyetheretherketone (PEEK). As discussed in greater detail below, compared to medical grade titanium and PEEK, silicon nitride significantly inhibits in vitro and in vivo bacteria colonization, and bio-film formation. Silicon nitride also exhibits a much lower live count and live to dead ratio for bacteria during studies.

It has also been demonstrated that silicon nitride materials provide significantly greater adsorption of vitronectin and fibronectin, which proteins are known to decrease bacteria function, than titanium, PEEK, and PEKK. It is thought that these properties will be very useful in biomedical implants of all types by significantly reducing the possibility of infection. This may be accomplished by, for example, preventing or disrupting bacterial formation on/in the implant and/or killing bacteria that have been transferred to the implant.

Without being limited by theory, it is thought that the higher adsorption of proteins that characterizes silicon nitride may facilitate the inhibition of bacteria growth and promote stem cell differentiation to osteoblasts. This preferential adsorption may be a cause for silicon nitride's ability to decrease bacteria function. Again, without being limited by theory, the mechanisms for the enhanced antibacterial characteristics of silicon nitride may be a combination of its features. For example, its hydrophilic surface may lead to preferential adsorption of proteins that are responsible for reduced bacteria function. This effect may be enhanced by increasing the surface texture or roughness of a silicon nitride based implant or silicon nitride based coating on an implant made up of a different material. Because of these characteristics, silicon nitride also exhibits enhanced in vivo osteoconduction and osteointegration when compared with titanium, PEEK, or PEKK alone.

As discussed above, using a silicon nitride coating or filler on one or more regions of an implant's surface may be used, in some embodiments and implementations, to inhibit bacterial adhesion, while increasing/fostering adsorption of proteins necessary for healing and bone reformation. This same effect may, in other embodiments, be accomplished using monolithic silicon nitride as an implant.

In such embodiments, the surface of the ceramic implant may be engineered to provide for an increased degree of micro-roughness and surface texture to enhance these desirable properties. For example, in some embodiments, the micro-roughness—i.e., the texture of the surface in between the peaks and valleys typically measured by Ra values—may also, or alternatively, be increased by suitable texturing. In some implementations, the micro-roughness of the implant and/or coating may be increased by micromachining, grinding, polishing, laser etching or texturing, sand- or other abrasive-blasting, chemical, thermal or plasma etching, and the like. Micro-roughness may be measured by measuring the height of surface asperities using cut-off limits on a profilometer. This method may be used to selectively assess the roughness of a surface between the peaks and valleys. Alternatively, or additionally, the skewness and/or kurtosis could be measured. These measurements consider the deviation of the surface from what might be expected of a normal Gaussian distribution of surface roughness. Such surface engineering may also be performed on a silicon nitride coating, rather than on a monolithic silicon nitride or silicon nitride composite implant.

In some embodiments, the density of the silicon nitride material, or doped silicon nitride material, may vary throughout the implant, or throughout the portion of the implant made up of silicon nitride. For example, in spinal implant embodiments, the outermost layer, or a portion of the outermost layer, may be more porous, or less dense, than the core or center of the implant. This may allow for bone to grow into or otherwise fuse with a less dense portion of the implant, and the denser portion of the implant can be wear-resistant and may have a higher strength and/or toughness, for example.

In certain embodiments, one or more inner portions of the implant may have a relatively low porosity or non-porous ceramic, and thus exhibit high density and high structural integrity generally consistent with, and generally mimicking the characteristics of, natural cortical bone. And, by contrast, one or more of the surface coatings, layers, or linings formed at an outer surface of the implant can exhibit a comparatively greater or higher porosity that is generally consistent with and generally mimics the characteristics of natural cancellous bone. As a result, the higher porosity surface region(s), coating(s), or lining(s) can provide an effective bone ingrowth surface for achieving secure and stable bone ingrowth affixation of the ceramic portion of the implant (which, in some embodiments, comprises the entire implant) between a patient's vertebrae or another suitable location within the human body.

In some embodiments, the antibacterial behavior of other implant materials, such as polymeric, metallic, or ceramics, may be improved through the application of silicon nitride as an adherent coating. This coating may, in some implementations, be roughened or textured to provide for increased surface area of the silicon nitride material/coating. In other embodiments, monolithic silicon nitride implantable devices may be provided which may be subjected to similar surface engineering.

The surface roughness values disclosed herein may be calculated using the arithmetic average of the roughness profile (Ra). Polished silicon nitride surfaces may have a roughness of 20 nm Ra or less. However, as discussed in greater detail below, counterintuitively, the antibacterial properties of certain embodiments may be improved by roughening, rather than polishing, all or one or more portions of the surface of a silicon nitride ceramic or another similar ceramic implant. In some embodiments, a relatively rough surface may be created as part of the process of creating the material, such as during a firing stage, without further roughening or other surface engineering. However, in other embodiments, as discussed in greater detail below, the surface may be roughened to further increase the roughness beyond what would occur as a result of standard firing/curing alone. Thus, in some embodiments, the surface roughness may be greater than about 1,250 nm Ra. In some such embodiments, the surface roughness may be greater than about 1,500 nm Ra. In some such embodiments, the surface roughness may be greater than about 2,000 nm Ra. In some such embodiments, the surface roughness may be greater than about 3,000 nm Ra. In other embodiments, the surface roughness may be between about 500 nm Ra and about 5,000 nm Ra. In some such embodiments, the surface roughness may be between about 1,500 nm Ra and about 5,000 nm Ra. In some such embodiments, the surface roughness may be between about 2,000 nm Ra and about 5,000 nm Ra. In some such embodiments, the surface roughness may be between about 3,000 nm Ra and about 5,000 nm Ra.

In certain embodiments, metallic, polymeric, or ceramic implant substrates may be filled with a silicon nitride powder to form a composite. Non-limiting examples of filler silicon nitride powders include α-Si₃N₄, β-Si₃N₄, and β-SiYAlON powders. Non-limiting examples of metallic or polymeric biomedical implant substrates that may be filled with silicon nitride powder include poly-ether-ether-ketone (PEEK), poly-ether-ketone ketone (PEKK), poly(methylmethacrylate), poly(ethyleneterephthalate), poly(dimethylsiloxane), poly(tetrafluoroethylene), polyacrylic acids, polylactic acids, polycarbonates, polyethylene, polyurethane, titanium, Silver, Nitinol, Copper, and/or related alloys. In various embodiments, a PEEK implant may be filled with α-Si₃N₄, β-Si₃N₄, or β-SiYAlON powders. In various other embodiments, a PEKK implant may be filled with α-Si₃N₄, β-Si₃N₄, or β-SiYAlON powders. The percentage of silicon nitride powder in the implant may range from about 1 weight percent (wt. %) to about 99 wt. %. In various aspects, the silicon nitride in the implant may range from about 1 wt. % to about 5 wt. %, from about 5 wt. % to about 15 wt. %, from about 10 wt. % to about 20 wt. %, from about 15 wt. % to about 25 wt. %, from about 20 wt. % to about 30 wt. %, from about 25 wt. % to about 35 wt. %, from about 30 wt. % to about 50 wt. %, from about 40 wt. % to about 60 wt. %, from about 50 wt. % to about 70 wt. %, from about 60 wt. % to about 80 wt. %, from about 70 wt. % to about 90 wt. %, and from about 80 wt. % to about 99 wt. %. In one embodiment, a PEEK implant may include up to about 30.4 wt. % β-Si₃N₄ or β-SiYAlON. In another embodiment, a PEKK implant may include up to about 30.4 wt. % β-Si₃N₄ or β-SiYAlON.

In still other embodiments, the percentage of silicon nitride powder in the implant may range from about 1 vol. % to about 99 vol. %. In various aspects, the silicon nitride in the implant may range from about 1 vol. % to about 5 vol. %, from about 5 vol. % to about 15 vol. %, from about 10 vol. % to about 20 vol. %, from about 15 vol. % to about 25 vol. %, from about 20 vol. % to about 30 vol. %, from about 25 vol. % to about 35 vol. %, from about 30 vol. % to about 50 vol. %, from about 40 vol. % to about 60 vol. %, from about 50 vol. % to about 70 vol. %, from about 60 vol. % to about 80 vol. %, from about 70 vol. % to about 90 vol. %, and from about 80 vol. % to about 99 vol. %. In one embodiment, a PEEK implant may include up to about 15 vol. % β-Si₃N₄ or β-SiYAlON. In another embodiment, a PEKK implant may include up to about 15 vol. % β-Si₃N₄ or β-SiYAlON.

In some additional embodiments, the implant may be 3D printed. A 3D printed implant of the present disclosure may include PEEK or PEKK and silicon nitride, or combinations thereof. In some aspects, the method of 3D printing used may include fused filament fabrication or selective laser sintering. In some examples, the 3D printed implant may include PEEK and silicon nitride. In other examples, the 3D printed implant may include PEKK and silicon nitride. In some embodiments, the printing speed may be between about 1500 mm/min to about 3000 mm/min. In some additional embodiments, the bed temperature of the 3D printer may be about 300° C. In still further embodiments, the print temperature may be about 400° C.

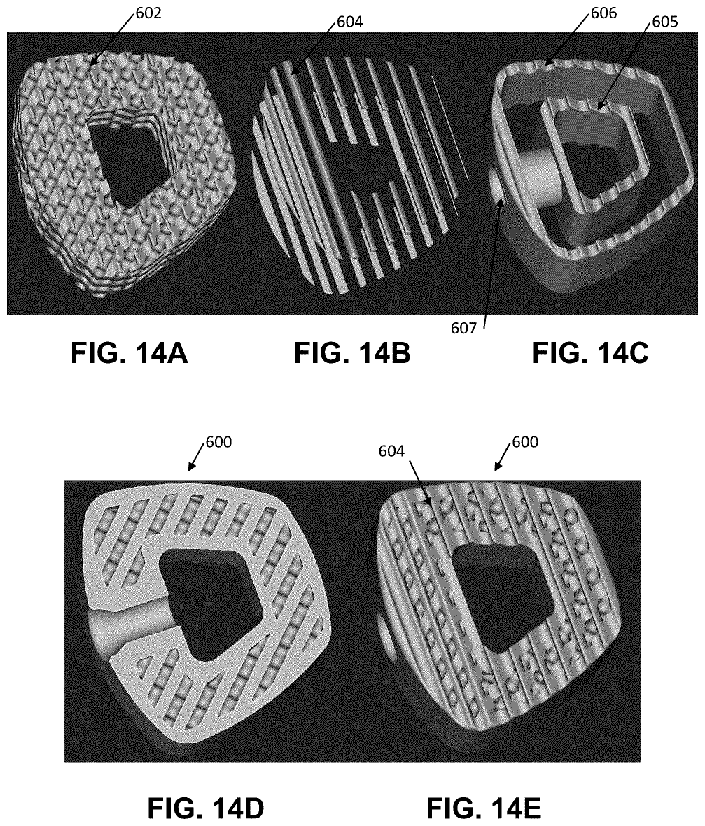

In some embodiments, the 3D printed implant may be a cervical spinal cage. FIGS. 14A-14E show an exemplary design of a 3D printed cervical spinal cage 600. In some aspects, the 3D printed cervical spinal cage 600 may include a diamond lattice structure 602 (see FIG. 14A), teeth 604 (see FIG. 14B), and an innerbody shell 605, outerbody shell 606, and support hole 607 (see FIG. 14C). The support hole 607 may connect between the innerbody shell 605 and the outerbody shell 606. The teeth 604 may be formed on an upper surface and/or a lower surface of the cervical cage 600. The upper and lower surfaces may be operable to contact a portion of one or more vertebra (e.g. cervical vertebra). Without being bound by theory, the addition of the teeth and the inner/outerbody shell and support hole increases the overall strength of the implant and improves its ability to osteointegrate with adjacent vertebrae. FIG. 14D shows a cross-section of the 3D printed cervical spinal cage 600 of FIG. 14E.

In some embodiments, the cross-section (e.g. as seen in FIG. 14D) of the 3D printed cervical spinal cage 600 may be between about 12 mm×12 mm to about 40 mm×40 mm. In some aspects, the length×width of the 3D printed cervical spinal cage may be between about 12 mm×12 mm to about 20 mm×20 mm, about 20 mm×20 mm to about 25 mm×25 mm, about 25 mm×25 mm to about 30 mm×30 mm, about 30 mm×30 mm to about 35 mm×35 mm, or about 35 mm×35 mm to about 40 mm×40 mm. In some additional aspects, the cross-sectional area of the 3D printed cervical spinal cage may be about 12 mm×12 mm, 15 mm×15 mm, 20 mm×20 mm, 25 mm×25 mm, 30 mm×30 mm, 35 mm×35 mm, or about 40 mm×40 mm. In further embodiments, the height of the 3D printed cervical spinal cage 600 may be between 5 mm and 30 mm. In some aspects, the height of the 3D printed cervical cage may be between about 5 mm and about 15 mm, about 10 mm and 20 mm, about 15 mm and about 25 mm, or about 20 mm and 30 mm. In some additional aspects, the height of the 3D printed cervical cage may be about 5 mm, 10 mm, 15 mm, 20 mm, 25 mm, or 30 mm.

In some embodiments, when the 3D printed cervical spinal cage 600 includes a diamond lattice structure 602, the diamond lattice structure 602 may have a unit cell with a length of about 3 mm. In some embodiments, the diamond lattice structure may comprise pores and struts. In some aspects, the diameter of the pores may be about 0.2 to 1.0 mm. In some examples, the diameter of the pores may be about 0.2 mm, 0.25 mm, 0.3 mm, 0.35 mm, 0.4 mm, 0.45 mm, 0.5 mm, 0.55 mm, 0.6 mm, 0.65 mm, 0.7 mm, 0.75 mm, 0.8 mm, 0.85 mm, 0.9 mm, 0.95 mm, or about 1.0 mm. In some additional aspects, the length of the struts may be about 0.5 to 1.5 mm. In some examples, the length of the struts may be about 0.5 mm, 0.55 mm, 0.6 mm, 0.65 mm, 0.7 mm, 0.75 mm, 0.8 mm, 0.85 mm, 0.9 mm, 0.95 mm, 1 mm, 1.05 mm, 1.1 mm, 1.15 mm, 1.2 mm, 1.25 mm, 1.3 mm, 1.35 mm, 1.4 mm, 1.45 mm, or about 1.5 mm.

In some aspects when the 3D printed cervical spinal cage 600 includes teeth 604 (see FIGS. 14B and 14E), the teeth 604 may have a longitudinal shape, such that the teeth are parallel to one another, separated by a gap. For example, the teeth 604 may have a thickness of between about 0.75 mm to about 1.5 mm. In some additional aspects, the teeth 604 may have a thickness of between about 0.75 mm to about 1.0 mm, about 1.0 mm to about 1.25 mmm, or about 1.25 mm to about 1.5 mm. In yet additional aspects, the teeth may have a thickness of about 0.75 mm, 0.8 mm, 0.85 mm, 0.9 mm, 0.95 mm, 1.0 mm, 1.05 mm, 1.1 mm, 1.15 mm, 1.2 mm, 1.25 mm, 1.3 mm, 1.35 mm, 1.4 mm, 1.45 mm, or about 1.5 mm.

In an embodiment, the SiYAlON and ß-Si₃N₄ materials may have added aluminum-oxide and yttrium-oxide. Without being limited to a particular theory, the functional surface chemistry of the implant may be enhanced by the additions of these oxide dopants.

In some embodiments, a polymeric implant filled with silicon nitride powder may improve the osteoconductivity and antibacterial activity of the implant compared to the implant without the silicon nitride filler. For example, a PEEK or PEKK implant filled with α-Si₃N₄, β-Si₃N₄, or β-SiYAlON may improve osteoconductivity and antibacterial characteristics of the implant compared to a monolithic PEEK or PEKK implant. In an embodiment, the surface of the implant filled with silicon nitride powder may be further modified with a surface roughness and may or may not further include a silicon nitride coating. Some of the methods disclosed herein may therefore provide for engineering of the surface roughness of silicon nitride ceramic filled implants in order to improve their antibacterial performance.

Without being limited to a particular theory, the addition of a relatively low fraction of α-Si₃N₄, β-Si₃N₄, or β-SiYAlON or a suitable mixture thereof may enhance in vitro osteoconductivity and antibacterial resistance of PEEK or PEKK. The silicon nitride filled PEEK or PEKK implants have substantially better results than other substrates without a silicon nitride filler. It was unexpected that a PEEK implant filled with α-Si₃N₄ exhibited an increased osteoconductivity and reduced antibacterial resistance while β-Si₃N₄ or β-SiYAlON had both an increased osteoconductivity and antibacterial resistance.

In some embodiments, metallic, polymeric, or ceramic substrates may be pre-engineered with a surface texture onto which a silicon nitride coating may be applied. This texture can range from as low as about 5 nanometers up to about 5,000 nanometers or more in average surface roughness (Ra). Alternatively, as another embodiment, the surface texture of the silicon nitride coating itself can be increased, exclusive of the surface roughness of the substrate, to obtain a similar Ra range and resulting antibacterial effect. Some of the methods disclosed herein may therefore provide for engineering of the surface roughness of monolithic silicon nitride ceramic implants in order to improve their antibacterial performance, and other methods disclosed herein may provide for engineering the surface roughness of layers or coatings applied to substrates made up of any other suitable material available for use in biomedical implants. Of course, in some implementations, surface engineering may be applied to both the substrate and the coating.

Increasing the surface roughness of the ceramic or ceramic filled implant can be accomplished using any number of known methods by those skilled in the art, including micromachining, grinding, polishing, laser etching or texturing, sand or other abrasive blasting, chemical etching, thermal etching, plasma etching, and the like.

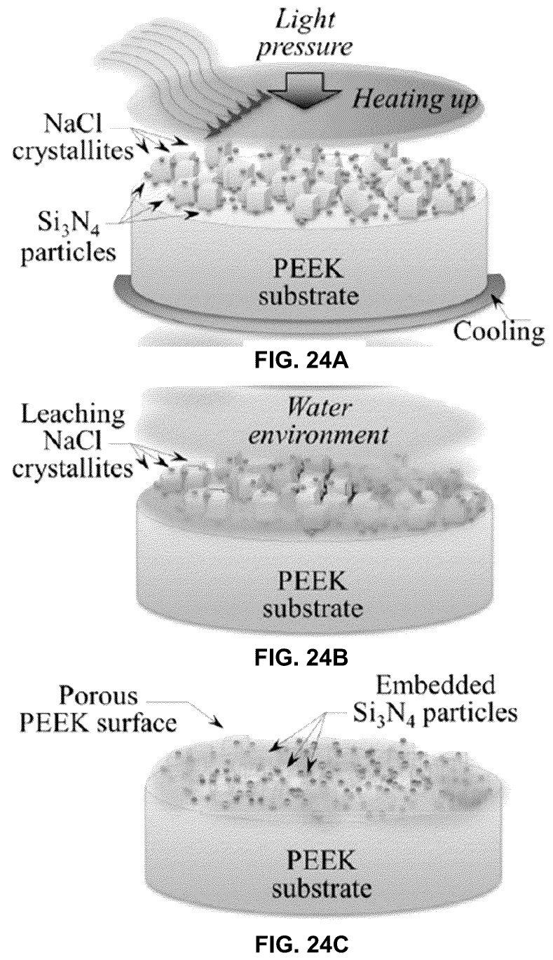

In some embodiments of the present disclosure, a porous PEEK surface embedded with silicon nitride may be formed by light pressing on a PEEK surface sprinkled with a mixture of coarse NaCl and fine silicon nitride grains, followed by leaching of the NaCl grains by distilled water at room temperature, as described in FIG. 24. In some aspects, the silicon nitride grains may have a diameter of about 5 μm to about 10 μm. The light pressing may be performed at a pressure of about 10 MPa with a hot plate at 340° C.

The inventive techniques disclosed herein, including but not limited to the silicon nitride coatings and roughened surface finishes, may be applied to any number and type of biomedical components including, without limitation, spinal implants, orthopedic screws, plates, wires, and other fixation devices, articulation devices in the spine, hip, knee, shoulder, ankle and phalanges, catheters, implants for facial or other reconstructive plastic surgery such as craniomaxillofacial implants, middle ear implants, dental devices, and the like. In aspects wherein the implant is a spinal cage, the implant may have properties that meet or exceed the minimum performance metrics outlined in ASTM F2077.

As illustrated in the Examples presented below, in comparison with titanium and poly-ether-ether-ketone (PEEK), silicon nitride significantly inhibits in vitro and in vivo bio-film formation and bacterial colonization, and shows much lower bacteria live/dead ratios for bacteria, including but not limited to Staphylococcus epidermidis (Staph. epi.), Staphylococcus aureus (Staph. aureus), Enterococcus, Pseudomonas aeruginosa (Pseudo. aeruginosa), and Escherichia coli (E. Coli). Silicon nitride also demonstrates significantly higher in vitro adsorption of three proteins (Fibronectin, Vitronectin, and Laminin) which can displace or inhibit bacteria growth and promote stem cell differentiation to osteoblasts.

In a clinical setting, bacteria are an ever-present menace, particularly when associated with surgical intervention and the introduction of foreign material into the human body, such as orthopedic, cardiac or dental endoprostheses. Microorganisms introduced during surgery tend to initially populate the sterile surfaces of implants. Bacterial adhesion to the biomaterial surface is the essential step in the development of an infection. The human body's defensive mechanisms are triggered if the implant is excessively colonized by bacteria. Chronic infections arise when the bacterial colony reaches a critical size and overcomes the local host defenses. When this occurs, the body tends to encapsulate the infection and reject the implant. Consequently, patients typically must undergo re-operation, removal of the implant, treatment of the infection, and replacement of the implant. Deep wound infections associated with common orthopedic surgeries can cost up to $100,000 or more for corrective treatment. The reduction in quality of life and the associated cost of treating infections represents a significant burden for present day medical care.

Various embodiments and implementations disclosed herein will therefore provide materials and methods that resist bacterial adhesion, colonization, and growth, which, as discussed above, often lead to chronic infections. The embodiments and implementations disclosed herein may also provide for enhanced in vivo osteointegration and increased bone growth in comparison to other common implants, such as those made up of only titanium and PEEK.

Factors influencing bacteria adhesion to biomaterial surfaces may include chemical composition, surface charge, hydrophobicity, and surface roughness or physical characteristics of the surface and/or coating of an implant. There are marked differences in the surface chemistry of metallic, polymeric, and ceramic implants. Metals typically have a thin protective oxide layer on their surfaces (typically less than about 25 nm in thickness). Polymers may also have oxide surfaces, but the oxides are typically part of longer chain carboxyl or hydroxyl groups. Both metallic and polymeric surfaces are often low in hardness, and therefore are easily abraded and highly sensitive to chemical attack and dissolution. Ceramics, such as silicon nitride, may also have oxide surfaces. However, unlike their metal counterparts, they are highly resistant to chemical and abrasive action.

Metallic and polymeric devices are also typically hydrophobic. Consequently, bacteria do not have to displace aqueous bodily fluids in order to adhere to the implant's surface. By contrast, ceramics, and silicon nitride in particular, are known to be hydrophilic. For instance, sessile water drop studies demonstrate that silicon nitride has higher wettability than either medical grade titanium or PEEK. This higher wettability is thought to be directly attributable to the hydrophilic surface of silicon nitride.

In order for bacteria to adhere to a hydrophilic surface, it must first displace the water that is present on the surface. Therefore, hydrophilic surfaces typically inhibit bacterial adhesion more effectively than do hydrophobic surfaces. It has also been shown that implant surface finish and texture play important roles in bacteria colonization and growth. Irregularities on the surface of typical polymeric or metallic implants tend to promote bacterial adhesion, whereas smooth surfaces tend to inhibit attachment and bio-film formation. This is true because rough surfaces have greater surface area and include depressions that provide favorable sites for colonization.

Counterintuitively, however, certain ceramic materials, including in particular silicon nitride, not only provide desirable antibacterial properties, but they also provide further enhanced antibacterial properties with increased, rather than decreased, surface roughness. In other words, silicon nitride surfaces of higher roughness appear to be more resistant to bacterial adhesion than smooth surfaces. This is precisely the opposite of what is observed for many other implant materials, such as titanium and PEEK. As referenced above and as discussed in greater detail below, compared to medical grade titanium and PEEK, silicon nitride has been shown to significantly inhibit in vitro bacteria colonization and bio-film formation, and it shows a much lower live count and a live to dead ratio for bacteria during studies. However, in studies between different types of silicon nitride, rough silicon nitride surfaces have been shown to be more effective in inhibiting bacterial colonization (rather than less effective as with most common implant materials) than polished silicon nitride (although both were much more effective in doing so than either titanium or PEEK).

Various embodiments and implementations will be further understood by the following Examples:

### Example 1

In a first working example, the abilities of biomedical implant materials to inhibit bacterial colonization were tested. The study included silicon nitride materials, biomedical grade 4 titanium, and PEEK. Four types of bacteria were included in the study: Staphylococcus epidermidis, Staphylococcus aureus, Pseudomonas aeruginosa, Escherichia coli, and Enterococcus.

Implant samples in the study were sterilized by UV light exposure for 24 hours and surface roughness was characterized using scanning electron microscopy. Bacteria were then inoculated on the surfaces of the samples and incubated for 4, 24, 48, and 72 hours.

Two methods were used to determine bacteria function at the end of each time period: (1) Crystal violet staining; and (2) Live/dead assay. Bacteria were also visually counted using a fluorescence microscope with image analysis software. The experiments were completed in triplicate and repeated three times. Appropriate statistical analyses were then compiled using Student t-tests.

For all bacteria, and all incubation times, the silicon nitride samples demonstrated lower bio-film formation, fewer live bacteria, and smaller live to dead bacteria ratios when compared with medical grade titanium and PEEK. Rough silicon nitride surfaces were even more effective in inhibiting bacterial colonization than polished surfaces. In addition, silicon nitride implants with polished or rough surfaces were both significantly better in inhibition of bacterial colonization than either titanium or PEEK.

Bio-film formation was also much higher for titanium and PEEK than for silicon nitride. For example, bio-film formation for Staphylococcus aureus on titanium was three times higher than polished silicon nitride after 72 hours of incubation and more than eight times higher than PEEK after 72 hours of incubation. And the results were even better using relatively rough silicon nitride having a surface roughness of about 1,250 nm Ra. Bio-film formation for Staphylococcus aureus on this rougher silicon nitride was less than half of that for the polished silicon nitride after 72 hours.

Live bacteria counts followed similar patterns. Live bacteria counts after 72 hours of incubation were between 1.5× and 30× higher for titanium and PEEK when compared with silicon nitride. And, again, rough silicon nitride outperformed polished silicon nitride. For example, for Pseudomonas aeruginosa, live bacteria count after 72 hours for rough silicon nitride (again, about 1,250 nm Ra) was about one-fifth of that for polished silicon nitride.

Live/dead bacteria ratios were similarly lowest for silicon nitride, and generally lower for rough silicon nitride than for polished silicon nitride. For example, live/dead ratios after 72 hours of incubation for E. coli on polished silicon nitride were over three times as high as titanium and about twice as high as PEEK. For rough silicon nitride, live/dead ratios were about six times as high for titanium and nearly three times as high for PEEK.

### Example 2

In this study, the ability of biomedical implant materials to adsorb common bone-forming proteins was tested. As with Example 1, rough silicon nitride, polished silicon nitride, medical grade titanium, and PEEK were tested. The proteins tested were fibronectin, vitronectin, and laminin. Enzyme-linked immunosorbent assays (ELISA) were performed for 20 minutes, 1 hour, and 4 hours. Fibronectin, vitronectin, or laminin were directly linked with primary rabbit anti-bovine fibronectin, anti-vitronectin, and anti-laminin, respectively. The amount of each protein adsorbed to the surfaces was measured with an ABTS substrate kit. Light absorbance at 405 nm on a spectro-photometer was analyzed with computer software. ELISA was performed in duplicate and repeated three different times per substrate.

For all incubation times, silicon nitride exhibited significantly greater adsorption of fibronectin and vitronectin when compared with titanium and PEEK. Silicon nitride also showed greater adsorption of laminin at 1 and 4 hours incubation in comparison to titanium and PEEK. Rough silicon nitride surfaces (approximately 1,250 nm Ra) were more effective in adsorption of proteins than polished silicon nitride surfaces. However, both silicon nitride surfaces were generally better than either titanium or PEEK, particularly for fibronectin and vitronectin. Without being limited by theory, it is thought that preferred adsorption of these proteins onto silicon nitride is a probable explanation for its improved bacterial resistance.

### Example 3

In this study, in vivo bone formation, inflammation, and infection of various implant materials were studied using a Wistar rat calvaria model. The study considered the strength of bone attachment to these materials. Rough silicon nitride, medical grade titanium, and PEEK were used in the study.

The study was conducted by implanting sterilized samples into the calvaria of two-year old Wistar rats using standard techniques. Another group of samples was inoculated apriori with Staphylococcus epidermidis and implanted into a second group of similar Wistar rats.

The animals were sacrificed at 3, 7, 14, and 90 days. Histology was quantified for the number of macrophages, bacteria, and bio-film proteins surrounding each of the implant materials. In addition, push-out tests were performed to determine bone attachment results and performance.

After 3 days using the non-inoculated samples, the titanium and PEEK implants were unstable, and thus no histology was able to be performed. The silicon nitride implants (surface roughness of approximately 1,250 nm Ra) exhibited about 3-5% bone-implant interface, as measured using microscopic linear analysis, and about 16-19% new bone growth in the surgical area, as measured using microscopic areal analysis, after 3 days.

After 7 days using the non-inoculated samples, the titanium and PEEK implants were unstable, and thus no histology was able to be performed. The silicon nitride implants, by contrast, exhibited about 19-21% bone-implant interface and about 28-32% new bone growth in the surgical area after 7 days.

After 14 days using the non-inoculated samples, the titanium implant exhibited about 7% bone-implant interface and about 11% new bone growth in the surgical area. The PEEK implant exhibited about 2% bone-implant interface and about 14% new bone growth in the surgical area. The silicon nitride implants, by contrast, exhibited about 23-38% bone-implant interface and about 49-51% new bone growth in the surgical area after 14 days.

After 90 days without inoculation, the titanium and PEEK implants exhibited about 19% and 8% bone-implant interface, respectively, and about 36% and 24% new bone growth, respectively. The silicon nitride implants again performed much better. These implants exhibited a bone-implant interface of about 52-65% and new bone growth of about 66-71%.

With the inoculated samples, all implants were too unstable to perform histology at 3 and 7 days. After 14 days, the titanium implant exhibited only about 1% bone-implant interface, 75% bacteria-implant interface (measured using microscopic linear analysis), about 9% new bone growth in the surgical area, and about 45% bacterial growth in the surgical area. PEEK exhibited essentially no bone-implant interface, about 2% new bone growth, and about 25% bacterial growth. The bacteria-implant interface with PEEK was unclear. The inoculated silicon nitride implants exhibited a bone-implant interface of about 3-13% after 14 days. New bone growth with the silicon nitride implants was about 25-28%, and bacterial growth was about 11-15%.

After 90 days, the inoculated titanium implant exhibited about 9% bone-implant interface, about 67% bacteria-implant interface, about 26% new bone growth, and about 21% bacterial growth. The PEEK implant exhibited about 5% bone-implant interface, about 95% bacteria-implant interface, about 21% new bone growth, and about 88% bacterial growth. The inoculated silicon nitride implants exhibited a bone-implant interface of about 21-25% after 90 days. New bone growth with the silicon nitride implants was about 39-42%, and there was no measurable bacterial-implant interface or bacterial growth after 90 days. In fact, there were no bacteria detected on the silicon nitride implants after 90 days.

Push-out strengths were also substantially better with the silicon nitride implants than with either the titanium or PEEK implants after all implantation times were measured, both with and without inoculation. After 90 days implantation without inoculation, push-out strengths for the silicon nitride implants were more than twice as high as titanium and more than two-and-α-half times as high as PEEK. With inoculation, silicon nitride push-out strengths were even better compared to titanium and PEEK for all implantation times. Silicon nitride push-out strengths were more than five times those of either titanium or PEEK. These results demonstrate substantial bone attachment for silicon nitride when compared to titanium and PEEK.

Push out strengths were measured by taking a sectioned portion of the calvaria including the implant and cementing the calvaria to wood blocks over a support plate. A load was then applied to the implant and the force required to dislodge the implant from the calvaria was measured.

The histology results further confirm the tested push-out strengths. As discussed above, significantly greater new bone growth was observed in the calvaria defect area for silicon nitride when compared with titanium and PEEK at all implantation times and under all inoculation conditions.

### Example 4

In this study, in vitro assessment of osteoconductivity of various implant materials were studied using a SaOS-2 cell line. The study considered the SaOS-2 cell proliferation on these materials. Silicon nitride filled PEEK (i.e., PEEK filled with 15 vol. % α-Si₃N₄, β-Si₃N₄, and β-SiYAlON powders) and monolithic PEEK substrate materials were used in the study.

The study was conducted by seeding SaOS-2 cells onto squares (5×10⁵ cells/ml) of each substrate material using standard techniques. After 24 hours, the cells were stained with Blue Hoechst 33342 and counted by fluorescence spectroscopy. Cell seeding was completed after 7 days. The cells were evaluated and counted by fluorescence spectroscopy and the substrate materials were evaluated using laser microscopy, Raman spectroscopy, and scanning electron microscopy (SEM).

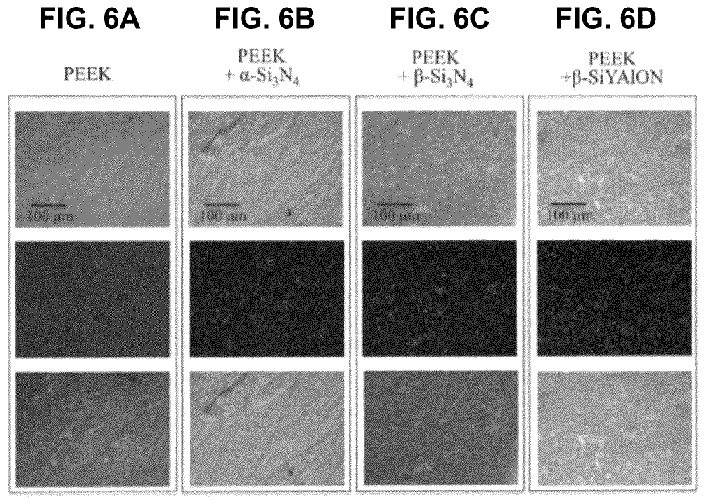

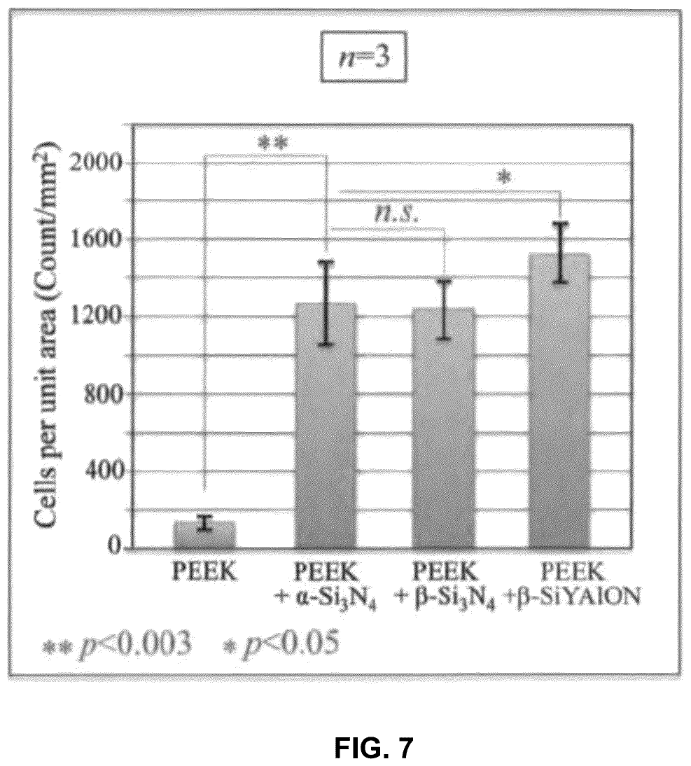

FIGS. 6A-6D show fluorescence spectroscopy images of the SaOS-2 cells on the various substrates. FIG. 7 is a graph of the results of cell counting based on the fluorescence microscopy. All composites showed a greater than 600% quicker SaOS-2 cell proliferation in vitro as compared to the monolithic PEEK. The PEEK with 15 vol. % β-SiYAlON demonstrated the greatest rate of proliferation with an increase of about 770% over the monolithic PEEK.

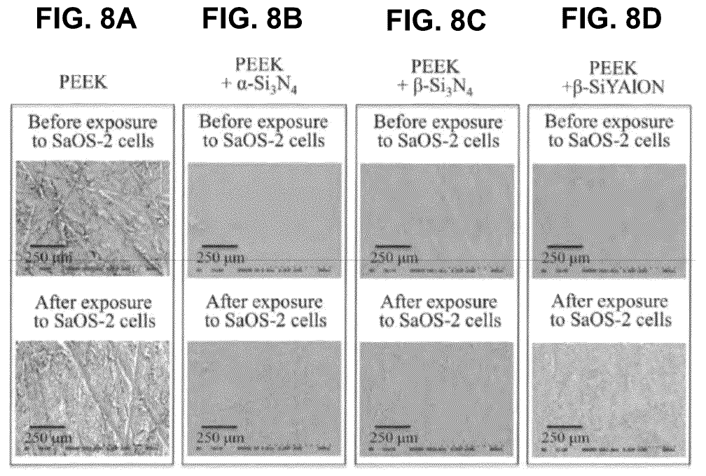

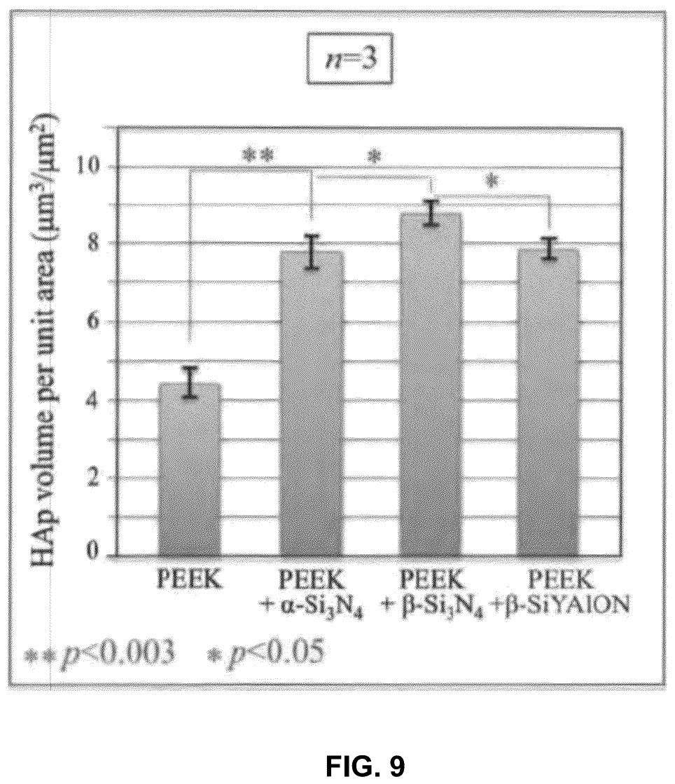

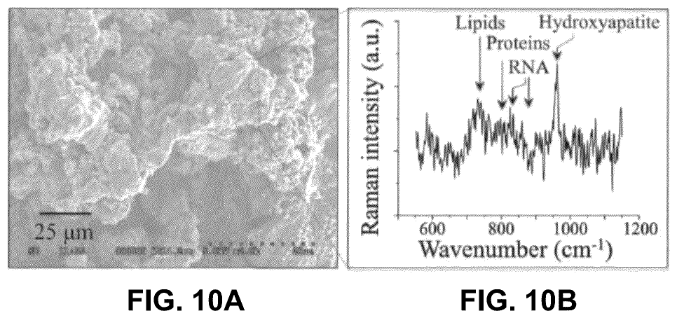

FIGS. 8A-8D show SEM images of the substrate materials before and after exposure to the SaOS-2 cells. FIG. 9 is a graph of the results of 3D laser microscopy of the substrate materials, showing the bony apatite volume. All composites behaved better than monolithic PEEK. PEEK with 15% Si₃N₄ exhibited an about 100% increase of in vitro osteoconductivity as compared to monolithic PEEK with SaOS-2 cells. FIGS. 10A and 10B show the results of Raman microprobe spectroscopy on β-SiYAlON filled PEEK after 7 days of being exposed to SaOS-2 cells. The surface protrusion after 7 days exposure to SaOS-2 cells was confirmed to be bony hydroxyapatite on all the composite samples.

All PEEK composites loaded with 15% Si₃N₄ (α- or β) or β-SiYAlON have shown a greatly improved SaOS-2 cell proliferation as compared with monolithic PEEK. All PEEK composites loaded with 15 vol. % Si₃N₄ (α- or β) or β-SiYAlON have shown significantly improved osteoconductivity with SaOS-2 cell line as compared with monolithic PEEK. The above results were confirmed by several different analytical tools and statistically validated.

### Example 5

In this study, in vitro assessment of antibacterial activity of various implant materials were studied using Staphylococcus epidermidis. Staphylococcus epidermidis (S. epidermis) is an important opportunistic pathogen colonizing on human skin inducing high probability of orthopedic device contamination during insertion. Costs related to vascular catheter-related bloodstream infections caused by S. Epidermidis are about $2 billion per year in US alone. Treatment with antibiotics is complicated by its capability of immune evasion, with high risk of chronic diseases.

The study considered the S. epidermis viability on these materials. Silicon nitride filled PEEK (i.e., PEEK filled with 15 vol. % α-Si₃N₄, β-Si₃N₄, and β-SiYAlON powders) and monolithic PEEK substrate materials were used in the study. S. epidermis was cultured (1×10⁷ CFU/ml) and then set in the samples of substrate materials in BHI Agar (1×10⁸/ml). After 24 hours, the bacteria and samples were assessed by Microbial Viability Assay (WST) and fluorescence spectroscopy by adding DAPI and CFDA and measuring concentration through absorbance at 450 nm.

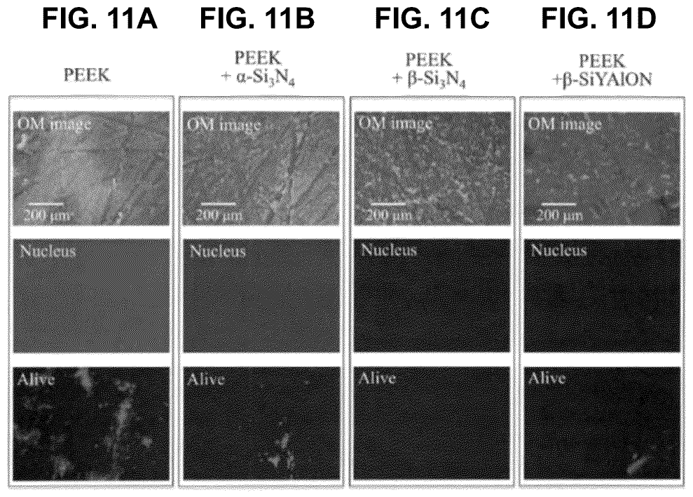

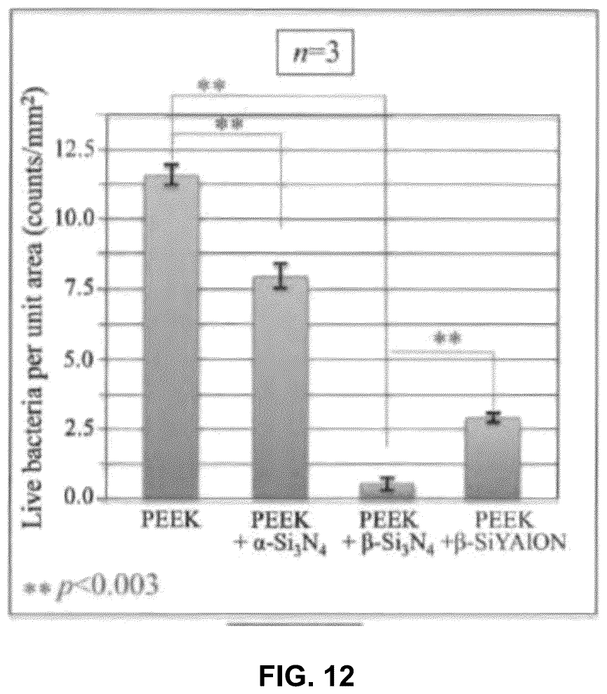

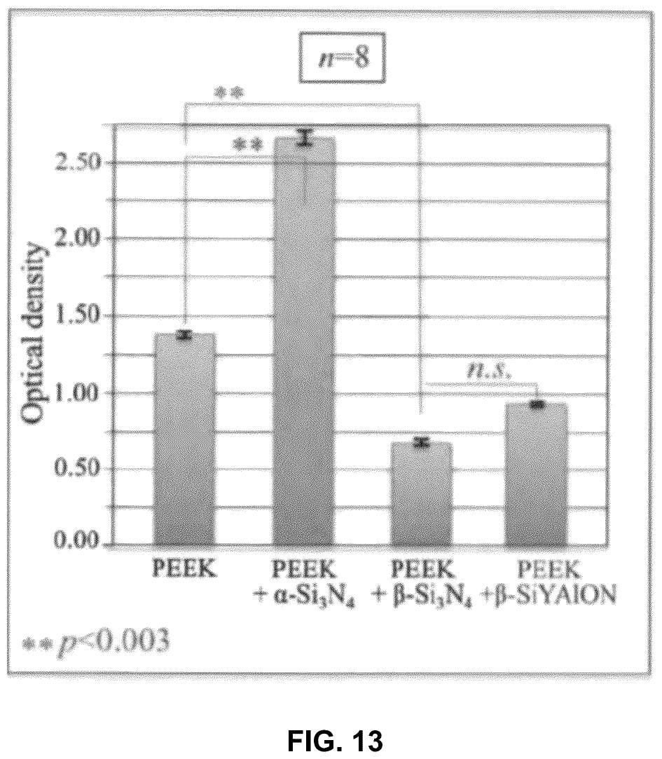

FIGS. 11A-11D show fluorescence microscopy images with DAPI (nucleus) and CFDA (alive) staining of S. epidermis on the various substrates. FIG. 12 is a graph of the results of CFDA/DAPI stained positive cells on the various substrates. PEEK with 15% β-Si₃N₄ showed about 1 order of magnitude increase of in vitro antibacterial resistance to S. epidermis as compared to monolithic PEEK. FIG. 13 is a graph of the results of the WST assay (absorbance at 450 nm) for each of the substrates. PEEK with 15 vol. % β-Si₃N₄ showed about a 100% increase of in vitro antibacterial resistance to S. epidermis as compared to monolithic PEEK.

PEEK composites loaded with 15 vol. % β-Si₃N₄ or β-SiYAlON have shown a greatly improved antibacterial resistance as compared with monolithic PEEK. The PEEK composite with 15 vol. % α-Si₃N₄ did not exhibit the same degree of antibacterial behavior as the other PEEK composites. The above results go clearly beyond a simple rule-of-mixture improvement and show how a relatively low fraction of β-Si₃N₄ phase could at least lead to 100% improved antibacterial resistance as compared to monolithic PEEK.

The results in each of the Examples discussed above suggest that, compared to medical grade titanium and PEEK, silicon nitride results in a substantially better inhibition of in vitro bacterial colonization and bio-film formation, and results in a much lower live to dead ratio for all studied bacteria at all incubation periods. Silicon nitride also demonstrates significantly higher in vitro adsorption of three proteins which may inhibit bacteria growth and promote stem cell differentiation to osteoblasts. This preferential adsorption correlates with, and may be a causative factor in, silicon nitride's ability to decrease bacterial function. Silicon nitride also exhibits enhanced in vivo osteogenesis and osteointegration and demonstrates significant resistance to bacteria compared to monolithic titanium and PEEK.

The studies discussed in the Examples also tend to suggest that roughened silicon nitride implants generally outperform polished silicon nitride in terms of antibacterial function and/or bone growth and integration. These results suggest not only that monolithic silicon nitride implants and/or or other similar ceramic implants may be surface roughened in order to improve antibacterial function, but also that silicon nitride coatings may be applied to other implants (both silicon nitride and non-silicon nitride, such as metals, polymers, and/or other ceramics). Such coatings may be surface roughened to further improve antibacterial function and provide other desirable characteristics, as discussed above. Preliminary research also tends to indicate that increasing the surface roughness beyond the levels used in the Examples—i.e. about 1,250 nm Ra—may further increase the antibacterial function of the material. For example, in some such embodiments, the surface roughness may be greater than about 1,500 nm Ra. In some such embodiments, the surface roughness may be greater than about 2,000 nm Ra. In some such embodiments, the surface roughness may be greater than about 3,000 nm Ra. In other embodiments, the surface roughness may be between about 500 nm Ra and about 5,000 nm Ra. In some such embodiments, the surface roughness may be between about 1,500 nm Ra and about 5,000 nm Ra. In some such embodiments, the surface roughness may be between about 2,000 nm Ra and about 5,000 nm Ra. In some such embodiments, the surface roughness may be between about 3,000 nm Ra and about 5,000 nm Ra.

Some alternative ceramic materials, such as alumina and zirconia (ZrO₂) for example, have certain properties that are similar to those of silicon nitride. As such, it is thought that these ceramic materials, or other similar materials, may exhibit similar antibacterial and osteogenic effects. It is thought that those of ordinary skill in the art, after having had the benefit of this disclosure, may be able to identify such alternative materials. It is also thought that these ceramic materials, or other similar materials, may exhibit improvement in antibacterial function with increased surface roughness, as is the case with silicon nitride ceramics.

Additional embodiments and implementations will be further understood by the following drawings.

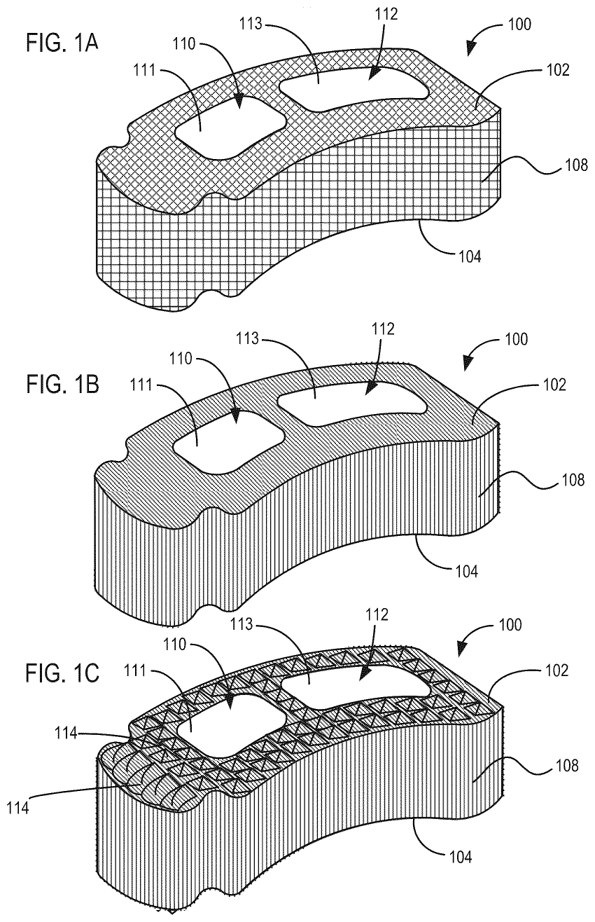

FIG. 1A depicts a spinal implant 100. Spinal implant 100 has relatively smooth top, bottom, and side surfaces (102, 104, and 108, respectively). Spinal implant 100 may comprise a silicon nitride ceramic material or another similar ceramic material. Spinal implant 100 also comprises two openings 110 and 112 extending through the top and bottom surfaces of the implant. In some embodiments, spinal implant 100 may comprise a doped silicon nitride material, as described in greater detail above. One or more of the surfaces of spinal implant 100 may be roughened or textured to provide for increased surface area of the silicon nitride material making up the surface(s). For example, one or more surfaces of spinal implant 100 may be roughened or textured by micromachining, grinding, laser etching or texturing, sand or other abrasive blasting, chemical etching, thermal etching, plasma etching, and the like.

FIG. 1B depicts spinal implant 100 after each of the exterior surfaces 102, 104 (surface not visible in the figure), and 108 has been roughened. As explained above, this surface roughening improves the antibacterial function and characteristics of the implant. One or more interior surfaces may also be roughened. For example, interior surfaces 111 and 113 that define openings 110 and 112, respectively, may also be roughened. The extent of roughening of the interior surfaces may be identical to, greater than, or less than, the roughening of exterior surfaces 102, 104, and 108, as desired.

FIG. 1C depicts spinal implant 100 having a plurality of surface features or teeth 114 on the top and bottom surfaces. Surface features 114 may help prevent or at least minimize migration of the implant once positioned within a patient's intervertebral space. Surface features 114 may be formed from the implant 100 before or after the surface roughening has taken place. Similarly, surface features 114 may, alternatively, comprise another material that is attached to the implant 100, again before or after surface roughening.

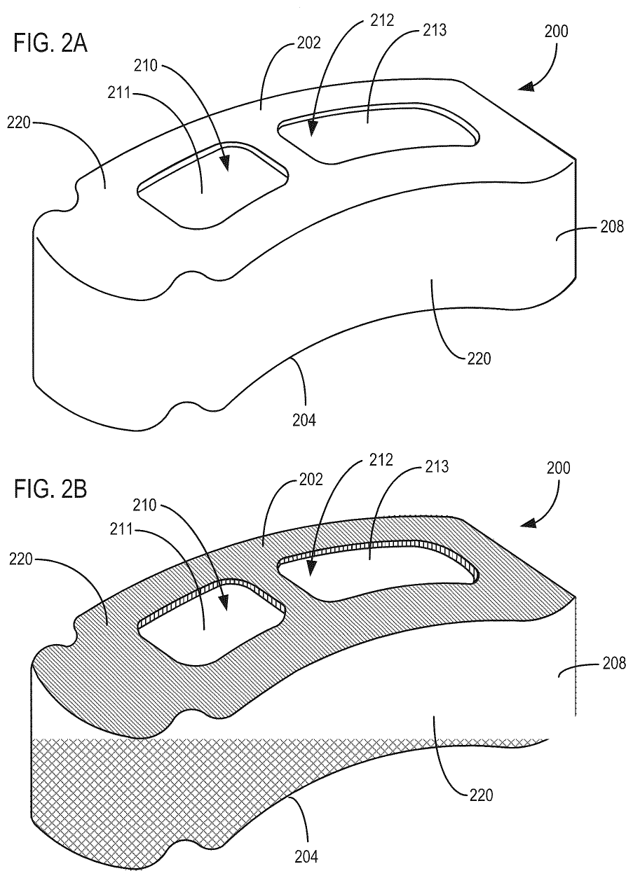

FIG. 2A depicts an alternative embodiment of a spinal implant 200. Spinal implant 200 may comprise any suitable material or materials, such as metals, polymers, and/or ceramics. Spinal implant 200 also comprises a coating 220. Coating 220 preferably comprises a silicon nitride or doped silicon nitride ceramic material, although it is contemplated that other ceramic materials having certain properties similar to silicon nitride may alternatively be used as a coating. Coating 220 may be applied to any surface exposed or potentially exposed to biological material or activity. For example, in the depicted embodiment, coating 220 is applied to top surface 202, bottom surface 204, side surface 208, and to interior surfaces 211 and 213 that define openings 210 and 212, respectively. Coating 220 may be applied to take advantage of the unique antibacterial properties and characteristics of silicon nitride discussed elsewhere herein. In some embodiments, the coating thickness can range from between about 5 nanometers up to about 5 millimeters. In some preferred embodiments, the coating thickness may be between about 1 micrometer and about 125 micrometers.

For example, because PEEK, which is very common in spinal implants, performs very poorly in a bacterial environment, silicon nitride ceramic coatings or layers (or another similar material) may be applied to a PEEK spinal implant to improve the antibacterial function of the implant and/or to provide other advantages as discussed in greater detail above. The coating(s) may be applied by any suitable methodology known to those of ordinary skill in the art, such as chemical vapor deposition (CVD), physical vapor deposition (PVD), plasma spraying, electro-deposition or electrophoretic deposition, slurry coating and/or high-temperature diffusion.

To further enhance the antibacterial characteristics of the implant, the coating 220, or one or more portions of the coating 220, may be surface roughened, as illustrated in FIG. 2B. The coating surface roughening may be applied to any and all portions of the implant that are or could be exposed to biological activity or material. For example, in the embodiment depicted in FIG. 2B, each of surfaces 202, 204, 208, 211, and 213 have been roughened or textured as described above. In some embodiments, the surface of the implant may be roughened or textured before the coating is applied, either in lieu of, or in addition to surface roughening or texturing on the coating.

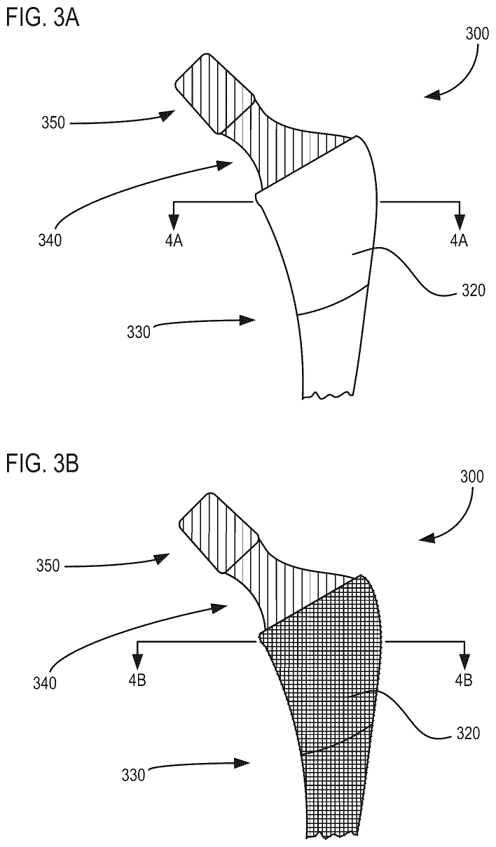

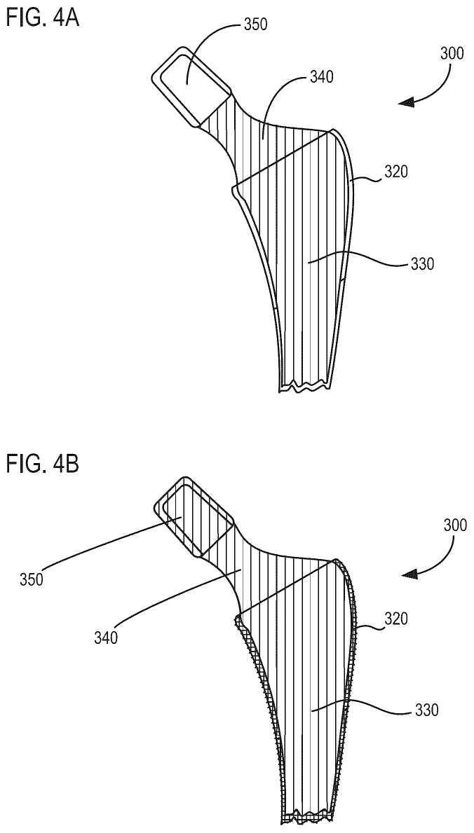

The principles, materials, and methods described herein may also be applied to other biomedical implants. For example, FIGS. 3A-3B and 4A-4B illustrate a hip implant 300 comprising a femoral stem 330 that is configured to be received within a patient's femur, a neck 340, and a modular acetabular head 350 configured to receive a ball joint (not shown) that will ultimately be positioned in an acetabular cup, or within a patient's natural acetabulum.

One or more coatings 320 may be applied to the femoral stem 330 of hip implant 300, as shown in FIG. 3A. In preferred embodiments, coating 320 comprises a silicon nitride ceramic material. In alternative embodiments, other portions of the implant may also be coated with a silicon nitride ceramic or another similar material. For example, coating 320 may also be applied to femoral stem 330, neck 340, and/or modular acetabular head 350, as desired.

In order to further enhance the antibacterial properties of the implant 300, one or more surfaces/portions of the implant 300 may be roughened and/or textured. For example, as shown in FIG. 3B, femoral stem 330, which comprises coating 320, may be roughened and/or textured after coating 320 has been applied. Alternatively, femoral stem 330 and/or any other desired region of implant 300 (or any of the other implants discussed herein) may be roughened and/or textured before coating 320 has been applied. In yet another alternative, one or more surfaces of the implant may be textured and/or roughened both before and after the antibacterial coating has been applied.

FIG. 4A is a cross-sectional view taken along line 4A-4A in FIG. 3A. As shown in this figure, coating 320 extends only along the femoral stem 330 portion of implant 300. However, as discussed above, in alternative embodiments, coating 320 may be applied to other portions of the implant as well (in some embodiments, the coating may be applied to the entire implant).

FIG. 4B is a cross-sectional view taken along line 4B-4B in FIG. 3B. This figure illustrates the surface of the femoral stem 330 of implant 300 after the roughening/texturing process has been completed.

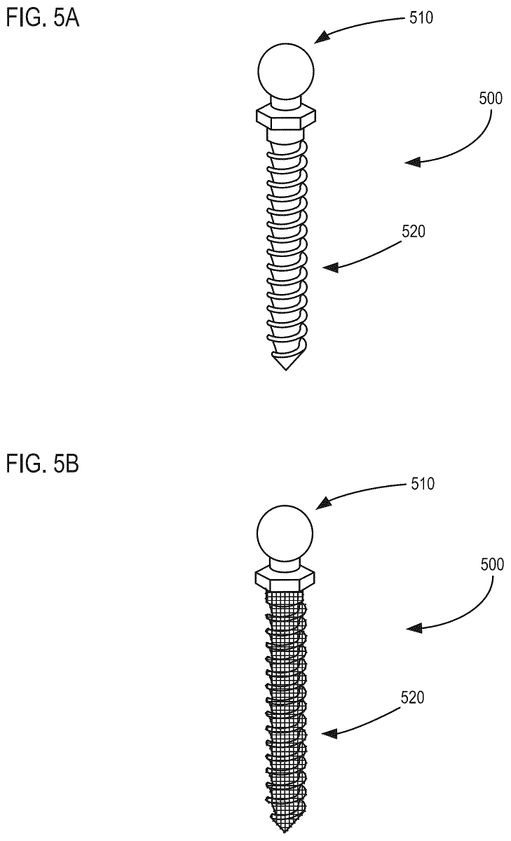

Still other alternative embodiments are depicted in FIGS. 5A and 5B. These figures illustrate a bone screw 500. Bone screw 500 may comprise a pedicle screw, for example. Bone screw 500 comprises a spherical head 510 and a threaded shaft 520. Bone screw 500, or one or more portions of bone screw 500, may comprise a silicon nitride ceramic material. One or more portions or surfaces of bone screw 500 may also be roughened or textured to improve antibacterial or other characteristics of the implant. For example, as shown in FIG. 5B, threaded shaft 520 has been roughened. Head 510 of screw 500 may remain smooth, or may be polished smooth, to provide for desired articulation within a spinal fixation system connector. However, for other embodiments, it may be desirable to roughen the surface of head 510 as well. This may provide for not only the improved antibacterial characteristics discussed herein but may also provide a desirable friction interface with another component of a spinal fixation system.

In other embodiments, a bone screw 500, or any of the other embodiments disclosed herein, may be comprised of another suitable material, such as titanium. In such embodiments, a silicon nitride coating may be applied to the implant rather than forming the entire implant from a silicon nitride material. As disclosed above, the coating and/or the undersurface of the coating (i.e., the surface of the original implant itself) may be roughened or textured to further improve antibacterial and other characteristics.

In still other embodiments, a bone screw 500, or any of the other embodiments disclosed herein, may comprise a biomedical material, such as a metal, ceramic, or polymer that includes a silicon nitride filler to form a composite, or that otherwise incorporate a silicon nitride material into the material used to form the implant. For example, silicon nitride may be used as a filler or otherwise incorporated into polymers, such as poly-ether-ether-ketone (PEEK), poly(methylmethacrylate), poly(ethyleneterephthalate), poly(dimethylsiloxane), poly(tetrafluoroethylene), polyethylene, and/or polyurethane. Silicon nitride may also be used as a filler or otherwise incorporated into other materials used to form other biomedical implants, such as metals, including titanium, silver, nitinol, copper, and related alloys, for example. As still another alternative, silicon nitride may be used as a filler or otherwise incorporated into other materials, such as oxide ceramics and cermets. By incorporating silicon nitride into other materials, it is expected that some of the antibacterial advantages and/or other advantageous properties described herein may be realized. Silicon nitride may also be incorporated into another materials to form a composite or used as part of one or more of the coatings described herein to increase antibacterial function.

It will be understood by those having skill in the art that changes may be made to the details of the above-described embodiments without departing from the underlying principles presented herein. For example, any suitable combination of various embodiments, or the features thereof, is contemplated.

Any methods disclosed herein comprise one or more steps or actions for performing the described method. The method steps and/or actions may be interchanged with one another. In other words, unless a specific order of steps or actions is required for proper operation of the embodiment, the order and/or use of specific steps and/or actions may be modified.

### Example 6

In this study, the response of KUSA-A1 mesenchymal progenitor cells to PEEK/Si₃N₄ composites was investigated to verify if the effects previously observed for monolithic ceramics can be observed also in polymer-matrix composites, namely upregulation of cell differentiation, inhibition of osteoclastogenesis and stimulation of the bone tissue production in vitro.

Samples were prepared as cylindrical discs with dimensions of Ø 12.7 mm and a thickness of 1 mm. Ti-alloy (ASTM F136-13, Ti6Al4V-ELI, Grade 23, Vincent Metals, Bloomington, MN, USA) and PEEK (ASTM D6262, Ketron©PEEK 1000, Quadrant EPP USA, Inc., Reading, PA, USA distributed by McMaster-Carr, Santa Fe Springs, CA, USA) discs were cut from commercially-sourced rod stok. Si₃N₄ discs were fabricated and were composed of 100% β-phase grains infiltrated with a minority amorphous Si—Y—Al—O—N phase. The composite material consisted of 15% (by volume) β-Si₃N₄ powder, fabricated from the same spray-dried feedstock and subjected to the same thermal cycling as the monolithic Si₃N₄ samples, dispersed within a PEEK (KetaSpire®, Solvay Specialty Polymers USA, LLC, Alpharetta, GA, USA) matrix. The composite was compounded and then extruded into rod stock using conventional techniques (Foster Corporation, Putnam, CT, USA).

The surface morphology of the samples was analyzed using a confocal scanning laser microscope (Laser Microscope 3D and Profile measurements, Keyence, VKx200 Series, Osaka, Japan). All images were collected at various magnifications ranging from 10× to 150×. Roughness values were measured on 25 randomized 100×100 mm square areas.

A field-emission-gun scanning electron microscope (JSM 700 1F Scanning Electron Microscope, JEOL, Tokyo, Japan) was used to obtain high magnification images of the morphology of the different samples following KUSA-A1 cell exposure. The instrument was also equipped with an Electron Dispersive X-ray Diffraction (EDS) probe. All images were collected at an acceleration voltage of 10 kV and magnifications between 250× and 20000×. Samples were sputter-coated (Cressington, Watford, UK) with a thin (20-30 Å) platinum layer before observation.

Fourier Transformed Infra-Red Spectroscopy (FT-IR) spectra were collected using a FT-IR Spectrometer (FT/IR-4000 JASCO, Tokyo, Japan) equipped with a Michelson 28 degree interferometer (corner-cube mirrors type). The aperture size was 200×200 mm² and the acquisition time was set to 30 seconds, for all samples. The instrument was operated using a dedicated software (Spectra Manager, JASCO, Tokyo, Japan). Signal averaging and deconvolution were performed using ORIGIN (OriginLab Corporation, Northampton, United States), and statistical analyses were obtained using “R”.

KUSA-A1 cells (JCRB, Osaka, Japan) were first cultured and incubated in medium consisting of 4.5 g/L of glucose DMEM (D-glucose, L-glutamine, phenol red, and sodium pyruvate, Nacalai tesque, Kyoto, Japan) supplemented with 10% fetal bovine serum.

The various samples were previously sterilized upon exposure to ethanol and put in the 24-well plate one by one. The cultured cells were then deposited on the samples in the well at the seeding concentration of 10⁵ cells/well. Cells were concentrated into 50 mL of solution and mildly deposited on the samples, then incubated for an hour. 1 mL of culture medium was added to each well.

An osteogenic medium was used after 24 hours. The medium consisted of Dulbecco's modified Eagle medium (DMEM) supplemented with nominal amounts of the following constituents: 50 mg/mL of ascorbic acid, 10 mM b-glycerol phosphate, 100 mM hydrocortisone, and 10% fetal bovine serum. All samples were incubated at 37° C. up to 10 days. The medium was changed a total of three times.

The cytotoxicity of the substrates was observed and compared analyzing the samples (N=3) using colorimetric assay based on water-soluble tetrazolium (Cell counting Kit-8, Dojindo, Kumamoto, Japan) which is based on the employment of a colori-metric indicator (WST-8). This later, upon reduction in the presence of an electron mediator, produced a water-soluble formazan dye which was directly proportional to the number of living cells. The resulted solutions were analyzed using micro-plate readers (EMax, Molecular Devices, Sunnyvale, CA, USA) upon collecting the OD value.

To mark the osteogenic differentiation, the membrane-bound enzyme ALP was detected on KUSA-A1 cells exposed to the different substrates (N=3) for 10 days in osteogenic medium. The ALP activity was monitored upon staining with the TRAP/ALP Stain Kit (Wako, Osaka, Japan) according to the manufacturer's instructions. ALP activity was assessed through direct pixel counting on optical micrographs using specific Image software (Rasband, W. S., ImageJ, National Institutes of Health, Bethesda, Maryland, USA).

The statistical significance of all performed experiments was checked by two-way analysis of variance (ANOVA). A p value <0.01 was considered statistically significant.

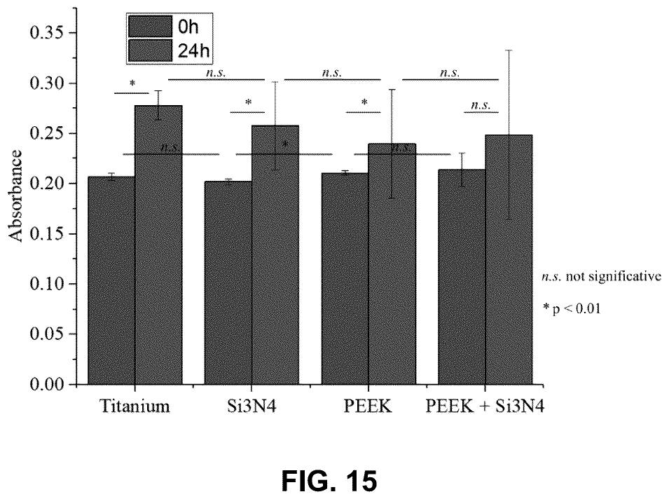

The values of WST as measured after 0 and 24 hours of in vitro testing are presented in FIG. 15. It was observed that while the initial values of absorbance are similar for all samples, with a narrow distribution. After ten days the values appear to be more scattered and the absorbance is higher for the titanium substrate and lower for the two PEEK-based materials, but with no statistical significance.

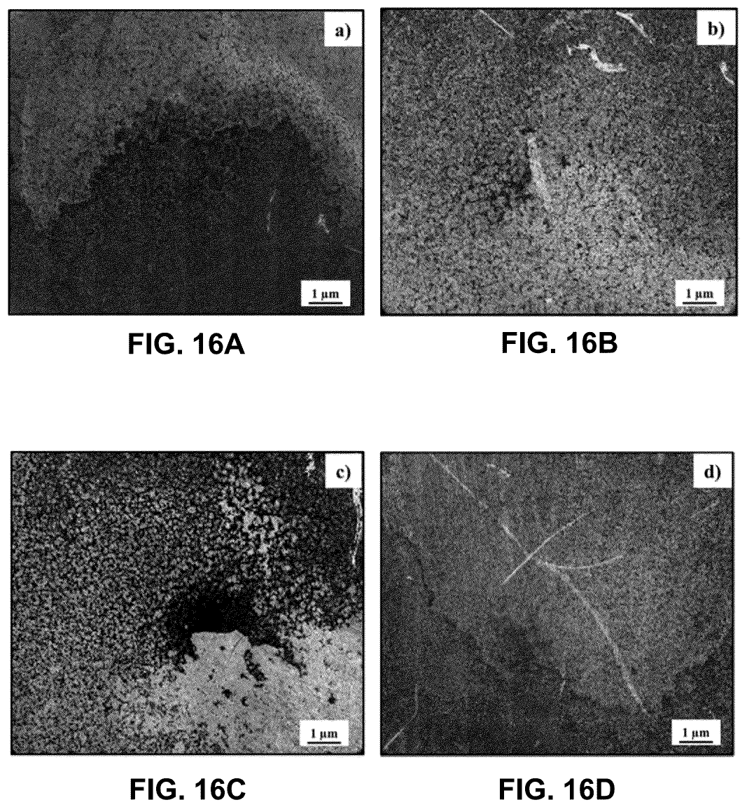

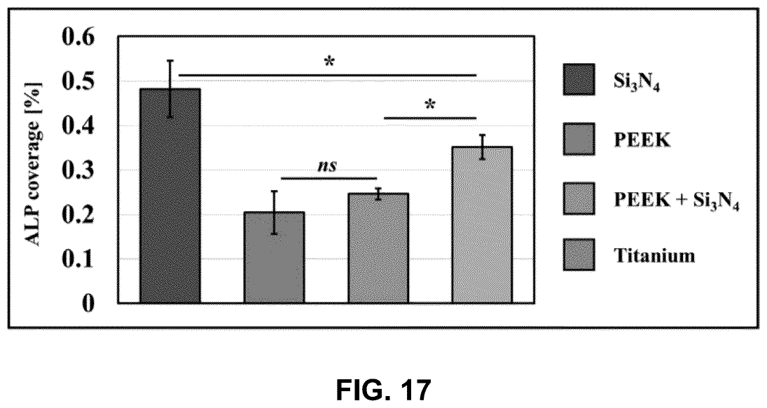

ALP-stained micrographs on silicon nitride, silicon nitride-reinforced PEEK, titanium alloy, and PEEK substrates are shown in FIGS. 16A-16D, respectively, after 10 days of testing. The level of ALP expression, an enzyme which represents osteogenic differentiation, was quantified at 10 days during osteo-genesis and reported in FIG. 17. The most intense staining was noted on Si₃N₄ compared with the others groups. Titanium alloy presented an unexpected, elevated level of ALP activity comparing with the two PEEK-based materials, but with a inhomogeneous distribution and an high concentration at the center of the sample. Comparing these last two samples, the untreated PEEK showed the lowest ALP concentration among the investigated samples, however, no statistical difference was shown between the two. The highest number of differentiated cells was observed by ALP on the surface of Si₃N₄, meaning that the ceramic reinforcement has a non-negligible effect on differentiation of KUSA-A1, an effect that seems to be strongly mitigated by the presence of a polymeric matrix (PEEK).

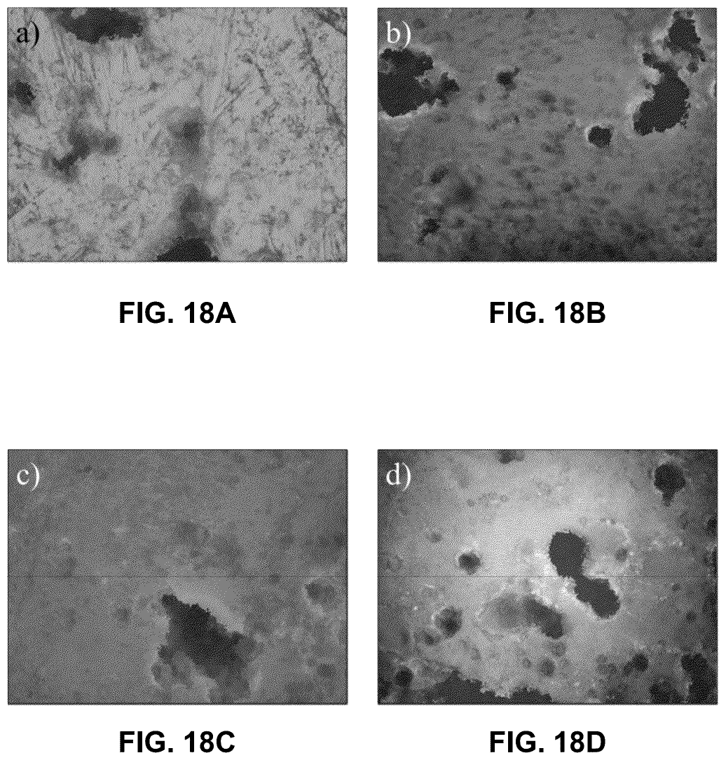

Laser microscope images obtained after 10 days indicate the presence of bone tissue formed on all samples. To correctly estimate the amount of bone tissue, the topographical and optical images were combined together to identify only the white areas (white is the natural color of hydroxyapatite) protruding from the surface. The results are marked in red in a few representative 1 Ox images on FIGS. 18A-18D. The amount of bone tissue (red areas) is lower on the titanium (FIG. 18A) and PEEK (FIG. 18C) samples, where it is mainly concentrated at the center of the sample, and higher on Si₃N₄ (FIG. 18B), with a more uniform distribution. The PEEK/Si₃N₄ composite (FIG. 18D) showed an intermediate behavior, with large and small red areas randomly distributed on the whole sample surface.

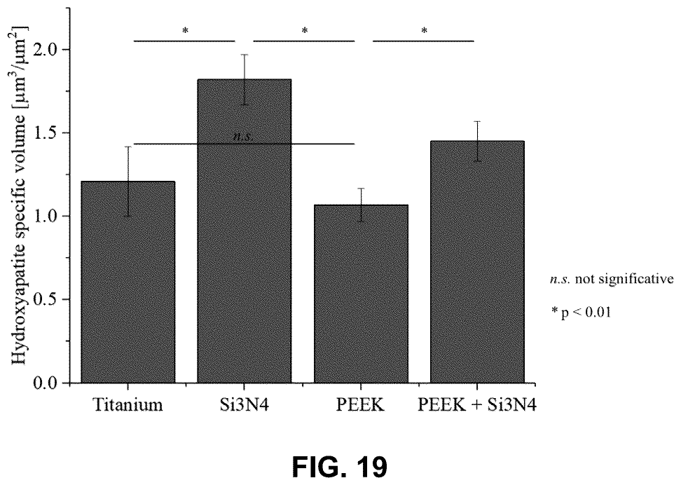

The specific volume of bone tissue per square micron of surface area, measured from the laser microscope images by dividing the protruding bone tissue volume by the total investigated area, is given in FIG. 19. As previously discussed, it can be observed that the highest amount of bone tissue was recorded on the surface of Si₃N₄, followed by the composite material, then titanium and lastly PEEK.

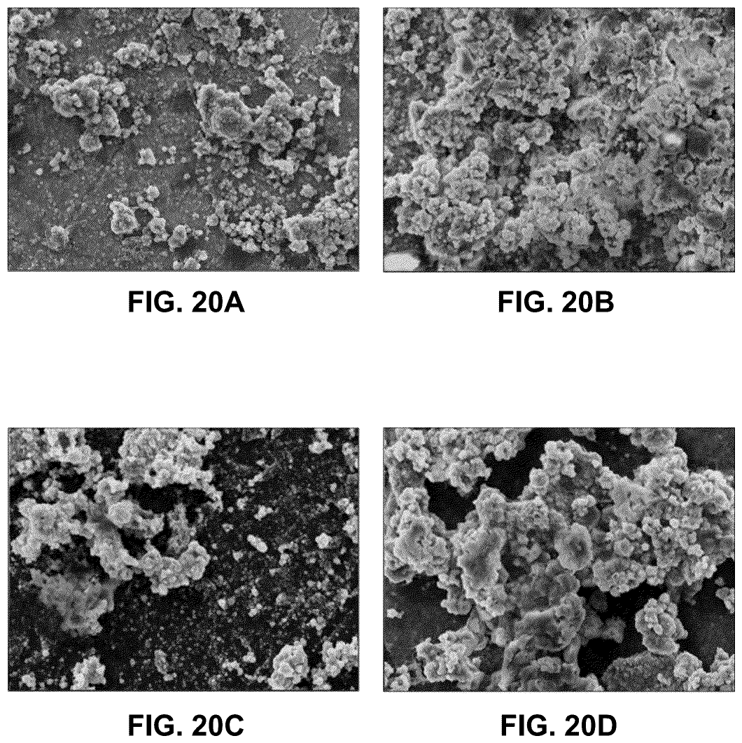

FIGS. 20A-20D show some representative SEM images obtained on the four different samples after 10 days of in vitro testing with the KUSA-A1 cells. Three different EDS maps have been superimposed on the greyscale image to show the locations of three elements, namely silicon (or titanium, in the case of the alloy), carbon and calcium and representing the presence of uncoated substrate, organic tissue and mineral apatite, respectively. It was observed that the amount of calcium is higher on the Si₃N₄ surface (FIG. 20B), followed by the composite (FIG. 20D) and finally the titanium (FIG. 20A) and PEEK (FIG. 20C) samples. While the biological carbon cannot be detected on the PEEK substrate, the presence of organic matrix can be clearly observed on both the Si₃N₄ and titanium samples. It was also observed that a relatively strong silicon signal was found on the composite surface, confirming the presence of reinforcing particles at, or close to, the outermost surface.

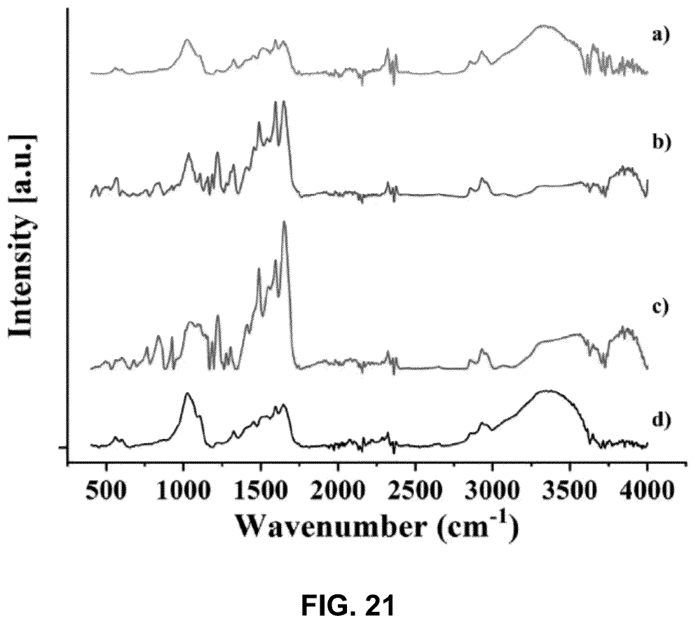

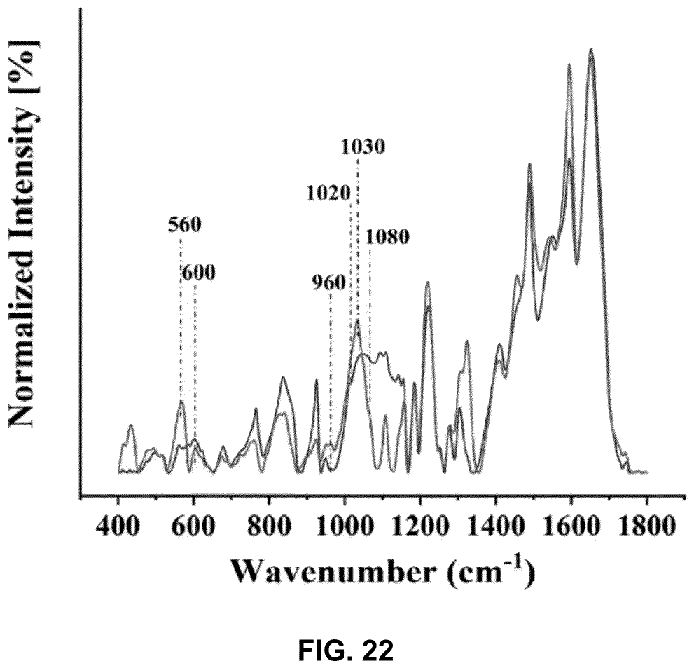

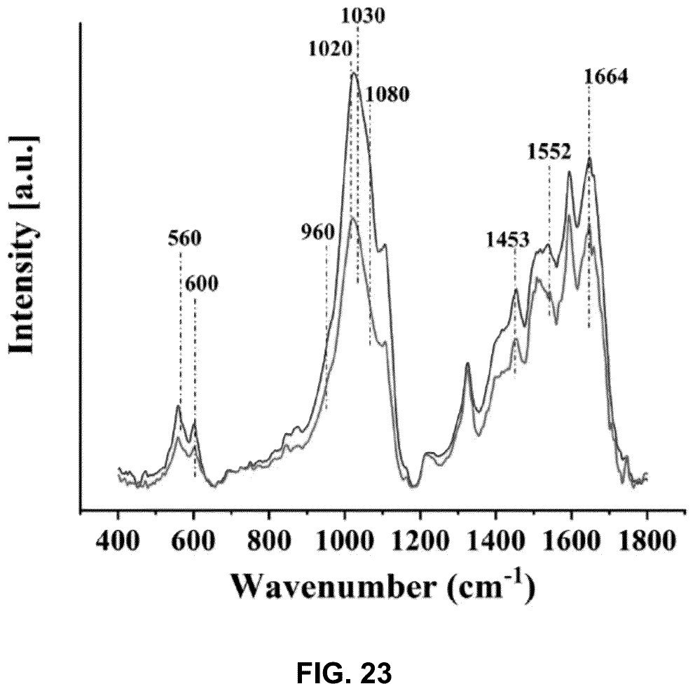

FIG. 21 shows the representative spectra of the four samples after KUSA-A1 testing, as obtained by FTIR (Table 1). It was observed that only untreated PEEK (FIGS. 21 and 22) average spectrum doesn't present evident bands related to the formation of mineral components on the surfaces. All of the bands are related to the vibrational modes of the substrate component. In the case of PEEK mixed with Si₃N₄ (FIGS. 21 and 22), after normalizing on peak referred to the substrate (925 cm⁻¹), the trend of the spectrum is different, in particular in the region between 900 and 1200 where clear bands are detectable. These latter, located at 960, 1030, 1040 and 1080 cm⁻¹ respectively, are related to vibrational modes of PO₄³⁻ of hydroxyapatite. There are other important features due to the presence of two bands at 560 and 600 cm⁻¹ related to vibrational modes of PO₄³⁻. About the average spectrum of KUSA-A1 exposed to Si₃N₄ (FIGS. 21 and 23), the area between 900 and 1200 cm⁻¹ clearly shows the formation of hydroxyapatite. The bands related to mineral components possess the highest intensity compared with the other samples. Similar trends for vibrational modes of CO₃⁻, amide I, and amide II bands at 1453, 1547 and 1664 cm⁻¹ compared the spectra collected on cells after exposure to Ti-alloy (FIG. 21) and both PEEK-based materials.

ALP staining (FIG. 17) and volumetric measurements of bone tissue formation (FIG. 19) confirmed once again the superior bio-logical activity of silicon nitride when compared to common structural biomaterials such as titanium and PEEK, as previously observed in literature.

PEEK, even if considered a bio-inert material, was outperformed even by titanium when considering the ALP osteogenic differentiation enzyme (FIG. 17) and showed the lowest amount of bone tissue formed on its surface after testing.

Results on PEEK-Si₃N₄ composites, on the other hand, suggest that the differentiation activity of PEEK cannot be improved just by adding limited amounts of reinforcing ceramic, as it seems to be regulated by the polymeric matrix. This hypothesis is supported by previous literature on mesenchymal stem cells differentiation on PEEK, when compared to osteoblasts.

The limited amount of cellular differentiation observed on Si₃N₄-reinforced PEEK still results in a total amount of bone tissue formed per square unit of surface (FIGS. 18A-18D and 19) comparable (if not superior) to titanium, which clearly shows higher levels of cellular differentiation in FIG. 17. This effect can be explained taking into account the osteoinductive effects of Si₃N₄. It has been previously postulated that silicon is leached from the ceramic particles in vitro and used by osteoblasts to nucleate hydroxyapatite, as also confirmed by an ex vivo spectroscopic experiment. Additionally, nitric oxide is also released from the ceramic surface, contributing to the regulation of cellular metabolism, differentiation and proliferation. Previous testing with osteosarcoma cell lines clearly demonstrated that Si₃N₄-PEEK composites exhibit higher levels of bone tissue formation and cellular proliferation when compared to titanium and bulk PEEK surfaces.

From the results of FIGS. 17, 18A-18D, and 19, and an analysis of the literature, it seems that the addition of Si₃N₄ strongly improves the osteoconductivity, as previously observed for SAOS-2 osteosarcoma cells, while the presence of the PEEK affects the cellular differentiation, neglecting the beneficial effects that were previously observed for bulk Si₃N₄ samples.

The FTIR spectroscopic analysis of FIGS. 21-23, confirm that more bone tissue, with higher degrees of mineralization, is formed in presence of Si₃N₄.

Thus, this study confirmed that Si₃N₄ positively interacts with mesenchymal stem cells, resulting in the formation of higher amounts of bone tissue with better degrees of mineralization. Monolithic PEEK samples showed poor osteogenic differentiation and low amounts of bone tissue formed after 10 days of cell exposure in vitro. Silicon nitride reinforced PEEK showed higher amounts of bone tissue formed.

### Example 7

In this study, the surface of a PEEK monolith was characterized, wherein the PEEK monolith was modified by heat-pressing a mixture of particulate NaCl and Si₃N₄ particles onto the PEEK. After water dissolution of the NaCl, a porous surface was left, with embedded Si₃N₄ particles.

Two sets of 12.7×3 mm discs (n=12 each) were prepared from commercially available bulk PEEK used to make spinal fusion cages (ASTM D6262, Ketron®PEEK 1000, Quadrant EPP USA, Inc. Reading P.A., USA; distributed by McMaster-Carr, Santa Fe Springs, CA, USA). Of 24 disks, eight were smooth control surfaces, and the other 16 underwent experimental treatments.

A roughened PEEK surface with coarse open pores (FIGS. 24A-24C) was made. The PEEK surface was sprinkled with coarse (150-300 μm) NaCl grains (n=8; NaCl-treated PEEK), while the remaining eight PEEK samples were similarly treated with a mix of NaCl coarse grains and finer (5-10 μm) R—Si₃N₄ particles (NaCl—Si₃N₄ PEEK). Sprinkled surfaces were light pressed (˜10 MPa) for 10 min with a hot plate (340° C.), while the other side of the PEEK samples was cooled on a cold plate (FIG. 24A). The heated PEEK partly entrapped the coarse NaCl grains or the NaCl/β-Si₃N₄ grain mixture. NaCl particles were removed by leaching in distilled water at room temperature (FIG. 24B), leaving coarse open pores. In the NaCl-β-Si₃N₄ PEEK samples, the surface retained a fine dispersion of ˜15 vol % β-Si₃N₄ particles (FIG. 24C).

The surface roughness of experimental groups was compared with a laser microscope coupled to 3-D imaging analysis software (VK-X200K series, Keyence, Osaka, Japan).

Human osteosarcoma (SaOS-2) cells were monitored with respect to cell adhesion and osteoconductivity tests. The SaOS-2 cells were cultured in an osteo-blast inducer medium consisting of 4.5 g/L glucose Dulbecco's modified Eagle medium (DMEM) (D-glucose, L-glutamine, phenol red, and sodium pyruvate) supplemented with 10% fetal bovine serum (FBS). Cell proliferation occurred on Petri dishes for 24 hours at 37° C. After achieving a final concentration of 5×10⁵ cells/mL, the cultured cells were deposited on the PEEK surfaces. Cell seeding in osteogenic medium (i.e. consisting of DMEM supplemented with 50 μg/mL ascorbic acid, 10×10⁻³ M β-glycerol phosphate, 100×10⁻³ M hydrocortisone, and 10% FBS) was followed by incubation for 7 days at 37° C. The medium was changed twice during the incubation period. Cell adhesion tests were repeated three times (n=3) for each sample; mean values were then plotted for comparison.

SaOS-2 cells were stained for fluorescence microscopy with Hoechst 33 342 (blue; nuclei) for 1 hour and then washed three times with 1 mL Tris Buffered Saline with Tween 20 (TBST) solution. Cell counts were performed in a fluorescence microscope (BZ-X700; Keyence, Osaka, Japan). The colorimetric assay of LDH Cytotoxicity test (LDH assay kit-WST; Dojindo, Kumamoto, Japan) was applied to quantitatively measure lactate dehydrogenase (LDH) released into the media from damaged SaOS-2 cells as a biomarker for cellular cytotoxicity and cytolysis.

For osteoconductivity testing, cells were seeded in DMEM osteogenic medium supplemented with 50 μg/mL ascorbic acid, 10×10⁻³ M β-glycerol phosphate, 100×10⁻³ M hydrocortisone, and 10% FBS. The test samples were then incubated for 7 days at 37° C. All experiments were repeated in triplicate (n=3); surfaces were examined with a Schottky-emission scanning electron microscope (SEM, Hitachi S-4300SE/N, Tokyo, Japan) at 15 kV and equipped with an energy dispersive x-ray spectrometer (EDX).

Gram-positive Staphylococcus epidermidis (S. epidermidis; 14990ATCC) were cultured at the Kyoto Prefectural University of Medicine in a brain heart infusion (BHI) agar culture medium with an initial concentration of 1.8×10¹⁰ CFU/mL. Bacterial concentration was diluted to 1×10⁸ CFU/mL using a phosphate buffered saline solution to obtain physiological ionic strength. The bacterial suspension was then transferred in 100 μL aliquots onto Petri dishes containing the substrate samples embedded in BHI medium. An incubation time of 24 hours at 37° C. was set under aerobic conditions followed by biological testing.

Bacterial metabolic activity was identified with the WST colorimetric assay (Microbial Viability Assay Kit-WST, Dojindo, Kumamoto, Japan), which employs the WST-8 indicator producing a water-soluble formazan dye upon reduction mediated by electrons. The amount of the formazan dye correlates with the number of live microorganisms. Solutions were analyzed using microplate readers (EMax, Molecular Devices, Sunnyvale, CA, USA) upon collecting optical density values.

Fourier transform infrared spectroscopy (FTIR) spectra were collected in time-lapse fashion after the osteoconductive tests. FTIR spectra were collected by means a high sensitivity spectroscope (Spectrum100FT-IR/Spotlight400; PerkinElmer Inc. Waltham, MA, USA). The spectral resolution of this equipment was 0.4 cm⁻¹. Average FTIR spectra targeting both bony hydroxyapatite and collagen grown by the SaOS-2 cells on the surface of different substrates were computed for each substrate from five independent measurements performed on n=4 samples. Pre-processing of raw data included baseline subtraction, smoothing, normalization, and fitting of the raw spectra using commercially available software (Origin 8.5, OriginLab Co., Northampton, MA, USA).

Experimental data were analyzed with respect to their statistical meaning by computing their mean value±one standard deviation and using one-way ANOVA for surface topography and two-way ANOVA with Tukey's post hoc analysis for biological assays; p≤0.01 was considered statistically significant and highlighted with two asterisks.

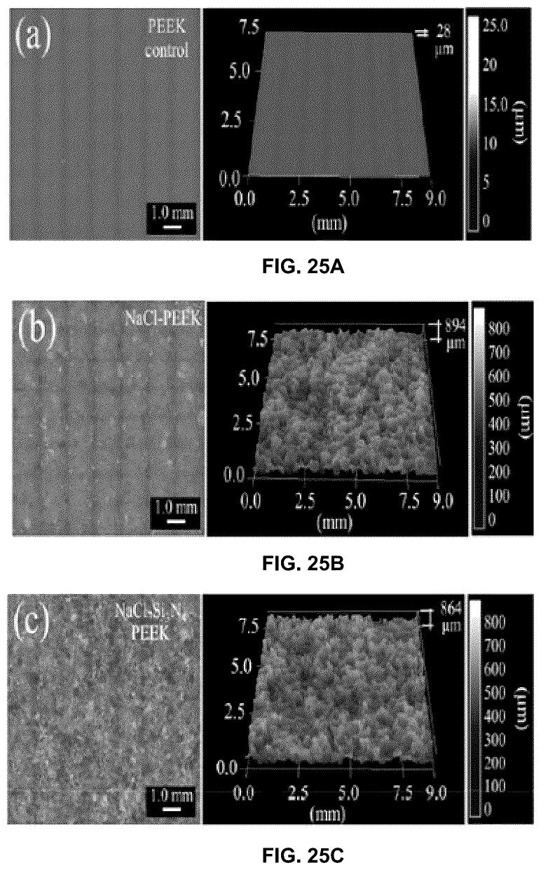

FIGS. 25A-25C show optical and laser microscopy images of the surfaces of smooth PEEK controls (FIG. 25A), NaCl-PEEK (FIG. 25B), and NaCl—Si₃N₄ PEEK (FIG. 25C). Highly porous morphologies were observed for both functionalized samples, when compared to smooth PEEK that showed its underlying machining profile.

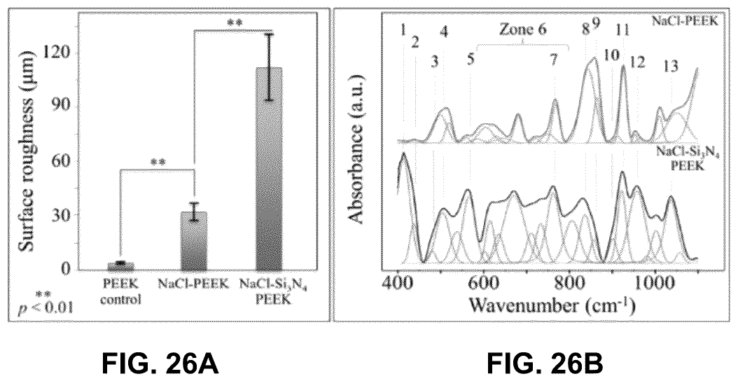

The results of topographic characterizations of the PEEK surfaces are shown in FIG. 26A. Both surface-treated samples were significantly rougher when compared to smooth PEEK. Of note, the mean surface roughness of the NaCl/Si₃N₄ group was the highest among the tested samples (˜115±27 μm), roughly three times the surface roughness of the NaCl-treated PEEK. Also the pore size of NaCl/Si₃N₄ samples was higher (250±56 μm) comparing with the NaCl-PEEK (170±45 μm); however, regarding pore depth, the two samples did not present any significant difference (417±87 μm versus 417±86 μm for the samples treated with NaCl/Si₃N₄ mixture and with NaCl only, respectively).

The presence of β-Si₃N₄ particles in PEEK was confirmed by FTIR spectroscopy. FIG. 26B shows average FTIR spectra recorded in the spectral interval 400-1100 cm⁻¹ on the surface of PEEK samples treated with only NaCl grains (upper spectrum) and with the NaCl/Si₃N₄ mixture (lower spectrum). A comparison of absorbances recorded from the two samples revealed one non-overlapping Band 1, and several partly overlapping but greatly enhanced bands from the β-Si₃N₄ ceramic phase, e.g. Band 5, Zone 6, and Band 12. FTIR data confirmed the presence of β-Si₃N₄ particles in the porous PEEK surface of the sample functionalized with the NaCl/R—Si₃N₄ mixture.

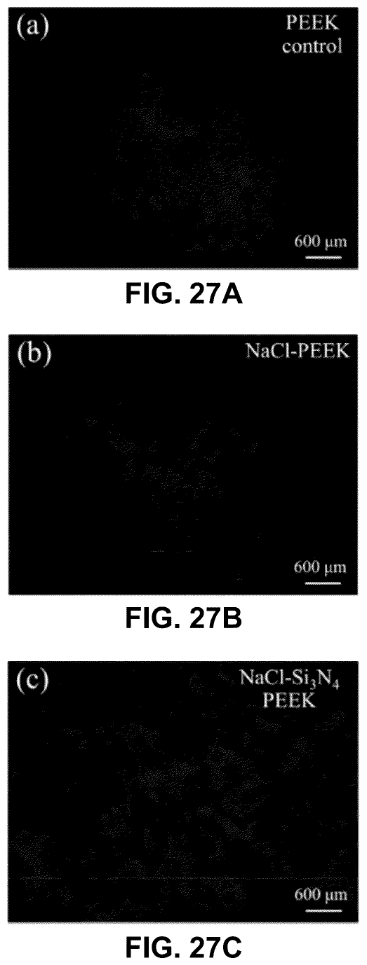

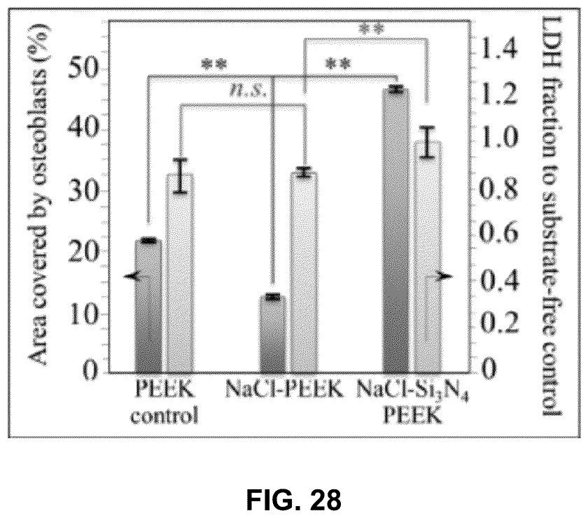

FIGS. 27A-27C show the results of fluorescence spectroscopy conducted on (Hoechst 33342) blue-stained SaOS-2 cell nuclei on: the smooth PEEK controls (FIG. 27A); NaCl-PEEK (FIG. 27B); and NaCl—Si₃N₄ PEEK (FIG. 27C). Observations were made after a one-week exposure of cells to the substrates. A quantification of the (blue) area covered by osteoblasts was made by image analysis over the entire surface of the disc samples. The results, which are plotted in FIG. 4D, show a significantly larger amount of cells on the PEEK surface functionalized with the NaCl—Si₃N₄ mixture. Interestingly, the amount of SaOS-2 cells proliferated on the smooth PEEK sample was 80% higher than that on PEEK samples functionalized with only NaCl grains. The cell proliferation results were confirmed by the output of LDH cytotoxicity tests, which is shown in FIG. 28.

The LDH plot was normalized to a control value obtained on SaOS-2 cells freely proliferating in substrate-free environment. The LDH value ˜1, for PEEK functionalized with NaCl—Si₃N₄ indicates a slight increase in substrate toxicity. In contrast, LDH values <1 were found for both smooth and NaCl-roughened PEEK with no statistical difference between these groups (labeled as n.s.). These data, and cell counts suggest that SaOS-2 cells were hardly stimulated to proliferate on PEEK surfaces alone, regardless of surface roughness. On the other hand, the higher amount of LDH detected on samples containing the NaCl—Si₃N₄ mixture is likely related to the higher amount of cells as compared to the samples functionalized with NaCl only.

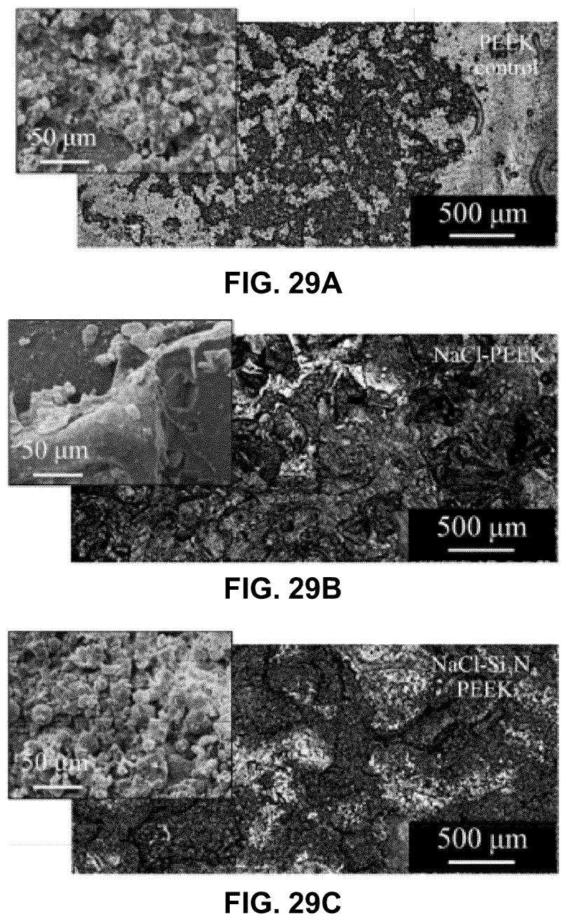

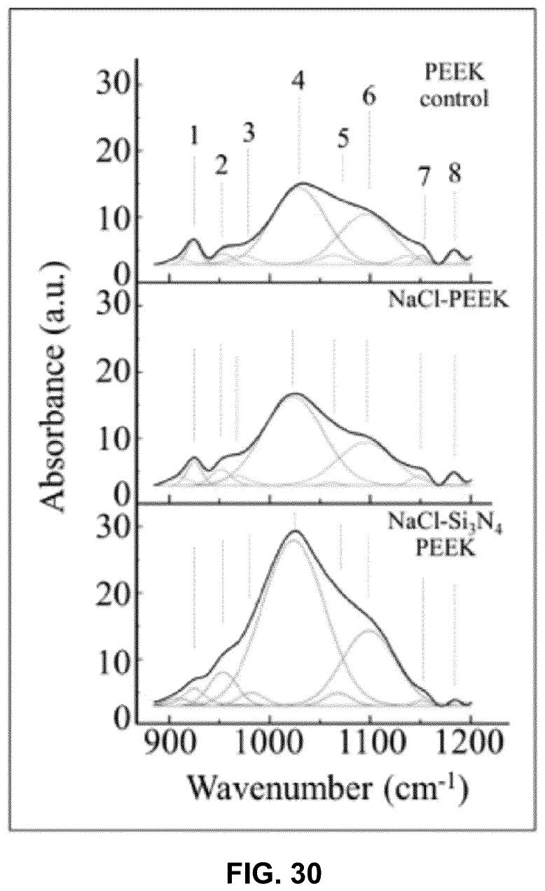

FIGS. 29A-29C show SEM images of the substrates after one-week exposure to SaOS-2 cells: smooth PEEK controls (FIG. 29A), NaCl-PEEK (FIG. 29B), and NaCl—Si₃N₄ PEEK (FIG. 29C). FIG. 30 shows FTIR spectra in the spectral region 900-1200 cm⁻¹ on the above three surfaces (upper, middle, and lower, respectively). Bands 2-7 are absorbance from bony apatite, while Bands 1 and 8 belong to the polymeric PEEK structure. A visual assessment on the low-magnification SEM images in FIGS. 29A-29C show that the amount of bony tissue (in dark contrast) deposited by osteoblasts on smooth PEEK was limited to isolated zones. This was also reflected in the low FTIR absorbance from mineral hydroxyapatite detected on this sample.

Higher amounts of bony apatite were found for NaCl-roughened PEEK, while the open pores in the NaCl—Si₃N₄ PEEK were completely filled with bony apatite (cf FIGS. 29B and 29C, respectively). FTIR absorbance values from mineral apatite showed improvements of 20% and 100% for NaCl-PEEK and NaCl—Si₃N₄ PEEK, respectively, over smooth PEEK (cf FIG. 30). The inset to the SEM images shows EDX maps of enlarged portions of the substrates; the green, blue, and red locate phosphorous, calcium, and carbon, respectively.

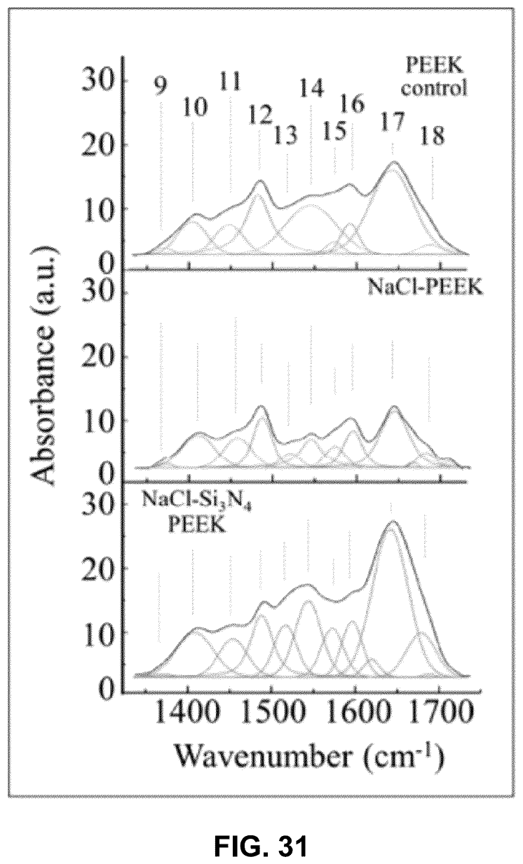

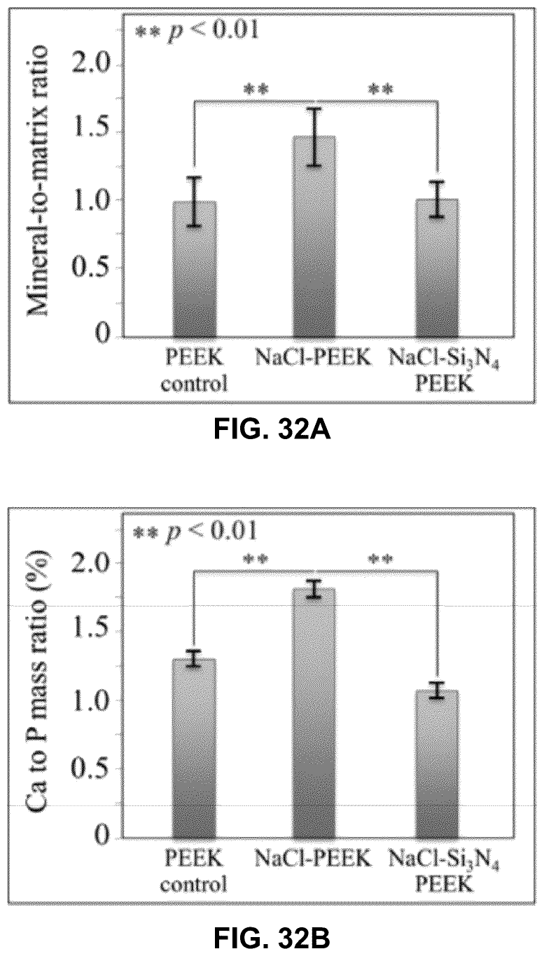

FIG. 31 shows the FTIR absorbance spectrum in the spectral region 1350-1750 cm⁻¹, which mainly represents the collagen structure of the bone tissue grown by osteoblasts. Bands labeled as Amide II and Amide I and related to the collagen structure have been identified. The main Amide I band at 1642 cm⁻¹, which is labeled as Band 17 in FIG. 31 and table 3, was taken as representative of the fraction of bone matrix. The mineral-to-matrix ratio was computed from the ratio between the apatite Band 4 (at 1025 cm⁻¹) and the Amide I Band 17 (at 1642 cm⁻¹) for the bony tissue grown by osteoblasts on the three different substrates investigated. The results are plotted in FIG. 32A. As seen, the bony tissue grown by SaOS-2 cells on the NaCl-PEEK scored a high mineral-to-matrix ratio, which is representative of a low bone quality and fragility. The smooth PEEK control and the NaCl—Si₃N₄ PEEK surfaces both produced low mineral-to-matrix ratios. These latter values were in the range of values reported for human bone tissue.

A comparison can also be made using EDX elemental data giving the Ca/P mass ratio, the latter data being shown in FIG. 32B. PEEK surfaces functionalized with NaCl—Si₃N₄ induced osteoblasts to produce bone tissue with the highest amount of phosphorus. Conversely, the NaCl-PEEK surface had the highest Ca/P mass fraction among the tested substrates. These data suggest a higher carbonate-to-phosphate ratio in this latter sample.

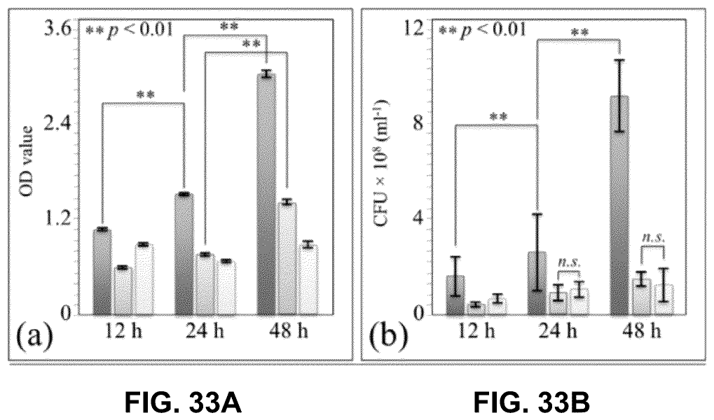

FIGS. 33A-33B show the results of OD measurement and CFU counting, respectively, on smooth PEEK, NaCl-PEEK, and NaCl—Si₃N₄ PEEK. The samples were contaminated with S. epidermidis bacteria at time zero and screened in time-lapse fashion after 12 hours, 24 hours, and 48 hours with respect to bacterial proliferation. Both plots consistently showed an exponential increase in bacterial population over time on the smooth PEEK surface. A less pronounced exponential increase was also observed for the OD value in NaCl-PEEK versus a nearly constant trend in OD value for NaCl—Si₃N₄ PEEK (with no statistical difference between 12 hours and 48 hours). However, the CFU counts showed only a slight (linear) increase (with no statistical difference) for both NaCl-PEEK and NaCl—Si₃N₄ PEEK surfaces (cf FIG. 33B).

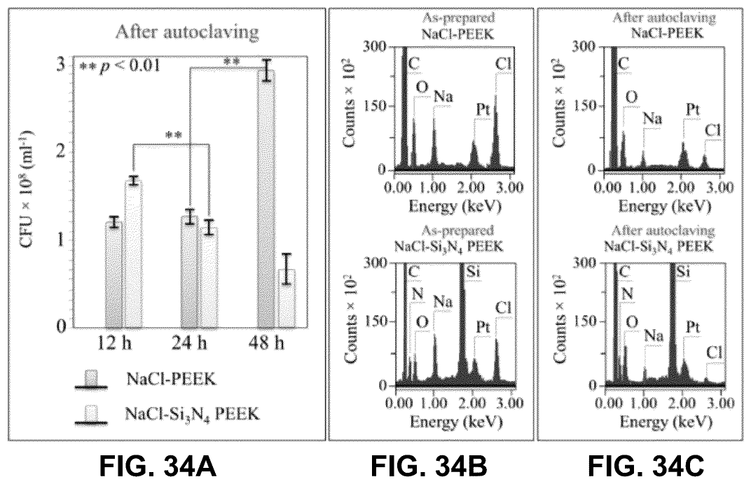

The bacterial proliferation experiments were repeated after preliminary autoclaving both surface-treated porous PEEK samples for 1 hour at 121° C. The CFU count was then carried out in order to clarify the nature of the improved bacteriostasis of both the porous PEEK samples versus the smooth PEEK (FIGS. 33A-33B). The results of this additional test are shown in FIG. 34A. Different from FIG. 33B, the NaCl-PEEK surface showed an exponential increase in bacterial population, while the NaCl—Si₃N₄ surface showed a clear, linear decrease in surviving bacteria. In order to clarify if any difference in chemical composition could be induced upon autoclaving, EDX spectra was collected on both NaCl-PEEK and NaCl—Si₃N₄ PEEK surfaces before and after autoclaving. The results of the EDX characterizations on these two functionalized surfaces are shown in FIGS. 34B and 34C, respectively. Both surfaces showed residual Na and Cl after water leaching, while the concentrations of these elements on both surfaces were reduced after autoclaving.

Tables 1 and 2 list, according to EDX analyses, the atomic fractions of the elements found on the NaCl-PEEK and NaCl—Si₃N₄ PEEK surfaces, respectively, before and after autoclaving. The results suggest that the as-prepared functionalized PEEK surfaces contained a non-negligible fraction of residual Cl, which was removed upon autoclaving. Based on the data in FIGS. 34A-34C, the antibacterial behavior of the NaCl-PEEK before autoclaving (cf FIGS. 33A-33B) may be related to retained Cl on the sample surface. Cl is a known antibacterial agent; its removal by autoclaving eliminated the antibacterial effect.

TABLE 1
Atomic fractions (at %) of the elements found on
the NaCl-PEEK surface before and after autoclaving,
as computed from the EDX spectra in FIG. 34B.

| | NaCl-PEEK (as prepared) | NaCl-PEEK (after |
|---|---|---|
| Element | (at %) | autoclaving) (at %) |
| C | 87.37 ± 0.28 | 87.00 ± 0.19 |
| N | 0.00 ± 0.00 | 0.00 ± 0.00 |
| O | 8.52 ± 1.04 | 11.38 ± 0.16 |
| Na | 1.51 ± 0.56 | 0.55 ± 0.04 |
| Si | 0.056 ± 0.01 | 0.05 ± 0.00 |
| P | 0.03 ± 0.01 | 0.03 ± 0.01 |
| Cl | 2.23 ± 0.73 | 0.68 ± 0.06 |
| Pt | 0.25 ± 0.01 | 0.29 ± 0.01 |
| Total | 100 | 100 |

TABLE 2
Atomic fractions (at %) of the elements found on the
NaCl—Si₃N₄ PEEK surface before and after autoclaving,
as computed form the EDX spectra in FIG. 34C.

| | NaCl-PEEK (as prepared) | NaCl-PEEK (after |
|---|---|---|
| Element | (at %) | autoclaving) (at %) |
| C | 71.32 ± 2.23 | 69.72 ± 3.49 |
| N | 12.96 ± 1.84 | 14.54 ± 2.98 |
| O | 7.7 ± 0.14 | 7.75 ± 0.32 |
| Na | 0.67 ± 0.05 | 0.15 ± 0.06 |
| Si | 6.40 ± 0.64 | 7.43 ± 0.85 |
| P | 0.00 ± 0.00 | 0.00 ± 0.00 |
| Cl | 0.68 ± 0.06 | 0.13 ± 0.06 |
| Pt | 0.24 ± 0.01 | 0.24 ± 0.01 |
| Total | 100 | 100 |

Osteointegration was less in NaCl-PEEK in the present study, probably because residual Cl discouraged cell proliferation (cf FIGS. 28 and 34B). In contrast, NaCl—Si₃N₄ promoted both osteoblast proliferation and apatite formation (cf FIGS. 28 and 29A-29C) while resisting gram-positive S. epidermidis (cf FIG. 34A). Throughout this specification, any reference to “one embodiment,” “an embodiment,” or “the embodiment” means that a particular feature, structure, or characteristic described in connection with that embodiment is included in at least one embodiment. Thus, the quoted phrases, or variations thereof, as recited throughout this specification are not necessarily all referring to the same embodiment.

Similarly, it should be appreciated that in the above description of embodiments, various features are sometimes grouped together in a single embodiment, figure, or description thereof for the purpose of streamlining the disclosure. This method of disclosure, however, is not to be interpreted as reflecting an intention that any claim require more features than those expressly recited in that claim. Rather, inventive aspects lie in a combination of fewer than all features of any single foregoing disclosed embodiment. It will be apparent to those having skill in the art that changes may be made to the details of the above-described embodiments without departing from the underlying principles set forth herein. The scope of the present invention should, therefore, be determined only by the following claims.

What is claimed is:

1. A method for forming a biomedical implant, the method comprising the steps of:
dispersing silicon nitride powder within a poly-ether-ketone-ketone (PEKK) substrate material to form a composite material; and
forming the composite material into a biomedical implant,
wherein the biomedical implant has improved bone-forming characteristics as compared to a monolithic PEKK implant.

2. The method of claim 1, wherein the silicon nitride powder is selected from α-Si₃N₄, β-Si₃N₄, β-SiYAlON, and combinations thereof.

3. The method of claim 2, wherein the silicon nitride powder comprises β-SiYAlON.

4. The method of claim 1, wherein the biomedical implant is a craniomaxillofacial implant.

5. The method of claim 1, wherein the biomedical implant is a spinal implant.

6. The method of claim 5, wherein the spinal implant comprises a diamond lattice structure between an innerbody shell and an outerbody shell.

7. The method of claim 5, wherein the spinal implant further comprises a support hole.

8. The method of claim 5, wherein the spinal implant further comprises teeth on an upper and/or a lower surface of the implant.

9. The method of claim 1, wherein the silicon nitride has a concentration in the implant of about 10 vol. % to about 20 vol. %.

10. The method of claim 1, wherein the composite material is formed into the biomedical implant using 3D printing.

11. A biomedical implant comprising:
a poly-ether-ketone-ketone (PEKK) substrate material; and
a powder comprising α-Si₃N₄, β-Si₃N₄, β-SiYAlON, or combinations thereof,
wherein the powder is dispersed within the substrate material, forming a composite material,
wherein the biomedical implant has improved bone-forming characteristics as compared to a monolithic PEKK implant.

12. The biomedical implant of claim 11, wherein the biomedical implant is a craniomaxillofacial implant.

13. The biomedical implant of claim 11, wherein the biomedical implant is a spinal implant.

14. The biomedical implant of claim 13, wherein the spinal implant comprises a diamond lattice structure between an innerbody shell and an outerbody shell.

15. The biomedical implant of claim 13, wherein the spinal implant further comprises a support hole.

16. The biomedical implant of claim 13, wherein the spinal implant further comprises teeth on an upper and/or a lower surface of the implant.

17. The biomedical implant of claim 11, wherein the concentration of silicon nitride in the implant is about 10 vol. % to about 20 vol. %.

18. The biomedical implant of claim 11, wherein the composite material is formed into the biomedical implant using 3D printing.

19. The biomedical implant of claim 11, wherein the biomedical implant has improved antibacterial characteristics as compared to a monolithic PEKK implant.

20. The biomedical implant of claim 11, wherein the biomedical implant has improved bone-forming characteristics as compared to a monolithic PEKK implant.

21. The biomedical implant of claim 20, wherein the improved bone-forming characteristics include improved osteoblast proliferation and improved apatite formation.
